(19) United States
(12) Patent Application Publication
(10) Pub. No.: US 2014/0057956 A1
(43) Pub. Date: Feb. 27, 2014

(54) COPOLYMERISED FATTY BODY, PREPARATION METHOD THEREOF AND USES OF SAME

(71) Applicants: Marc Balastre, Paris (FR); Chi-Thanh Vuong, Lognes (FR)

(72) Inventors: Marc Balastre, Paris (FR); Chi-Thanh Vuong, Lognes (FR)

(73) Assignee: RHODIA OPERATIONS, Aubervilliers (FR)

(21) Appl. No.: 14/114,243

(22) PCT Filed: Apr. 27, 2012
(86) PCT No.: PCT/EP2012/057813

§ 371 Date: Nov. 11, 2013

(30) Foreign Application Priority Data
Apr. 28, 2011 (FR) 1153634
Apr. 28, 2011 (FR) 1153636

(51) Int. Cl. A61K 47/14; A61K 31/4196

(52) U.S. Cl. CPC A61K 47/14; A61K 31/4196
USPC 514/383; 560/190; 514/785

(57) ABSTRACT

The invention relates to a copolymer having a backbone that is obtained by radical polymerisation of: a fatty body (A) comprising unsaturations and/or hydroxyl functions; and at least one monomer (B) including at least one function that can be polymerised by means of radical polymerisation and comprising either (i) at least one linear or branched alkyl chain having preferably between 16 and 44 carbon atoms, and more preferably at least 18 carbon atoms, e.g. at least 20 carbon atoms, in particular 22 carbon atoms or 44 carbon atoms, or (ii) a reactive function. The invention also relates to a method for modifying the rheological properties of an non-aqueous medium with the addition of a copolymer of the invention. The invention further relates to a composition formed totally or partially by a copolymer of the invention. Preferably, the composition is an emulsifiable composition. Furthermore, the invention relates to a composition comprising a copolymer of the invention and an emulsifying agent.

The present invention relates to the copolymerization of a fatty substance for in particular modifying its rheological properties, to the resulting copolymer and to the use thereof for modifying the properties, in particular rheological properties, of a nonaqueous medium.

Formulations based on nonaqueous, in particular nonpolar, media, for example oils, are used in many fields, such as agrochemistry, cosmetics, the oil industry, lubricating compositions, coating compositions, pharmacy, etc.

It is generally necessary to modify the rheology of these media in order to obtain the desired properties. Unlike aqueous formulations for which numerous effective rheological agents (thickener, dispersant, etc.) exist, it is more difficult to find rheological additives that are suitable over a wide temperature range for nonaqueous media.

Some technologies which use in particular inorganic materials (fumed silica, modified clays such as bentonites) are already employed for modifying the rheology of nonaqueous, especially nonpolar, media, in particular oils. Generally, depending on the desired functionality and the intended application, it is necessary to very precisely develop products resulting from each of these technologies. Nevertheless, for all these applications, it would be useful to be able to provide a product which is easy to use, which has a reasonable cost and which confers on nonaqueous, in particular nonpolar, media, in particular oils, a pseudoplastic behavior with the presence of a rheological threshold, over a wide temperature range (including under drastic storage conditions, in particular in terms of duration and temperature).

It is therefore of interest to provide a compound and a process for modifying the rheology of nonaqueous, in particular nonpolar, media, in particular oils, which are easy to use, which have a reasonable cost and which confer on said nonaqueous, in particular nonpolar, media, in particular said oils, a pseudoplastic behavior with the presence of a rheological threshold, over a wide temperature range.

An objective of the present invention is to provide a copolymer which makes it possible in particular to modify the properties, in particular the rheological properties (presence of a rheological threshold and/or modification of the viscosity, advantageously an increase in the viscosity, and/or modifications of the gelling properties), of a nonaqueous, in particular nonpolar, medium, in particular of oils.

Another objective of the invention is to provide a simple process for modifying the rheology (presence of a rheological threshold and/or modification of the viscosity, in particular increase in the viscosity, and/or modification of the gelling properties) of nonaqueous, in particular nonpolar, media, in particular of oils.

Another objective of the present invention relates to a process for modifying the properties, in particular the rheological properties (presence of a threshold and/or modification of the viscosity, in particular increase in the viscosity, and/or modification of the gelling properties), of a fatty substance.

Other objectives will become apparent on reading the description of the present invention.

In the context of the invention, the term “fatty substance” is intended to mean any compound containing a linear or branched aliphatic chain comprising at least 4 carbon atoms, for example at least 6 carbon atoms, for example at least 8 carbon atoms, for example at least 10 carbon atoms. It can be a lipophilic or amphiphilic compound. For example, the fatty substance may be selected from oils, or derivatives thereof, that are liquid at ambient temperature, in particular between 15 and 30° C., for example at 25° C.; fats, or derivatives thereof, that are pasty or solid at ambient temperature, in particular between 15 and 30° C., for example at 25° C.; waxes, or derivatives thereof, that are solid at ambient temperature, in particular between 15 and 30° C., for example at 25° C.

The fatty substance according to the invention is generally water-immiscible. A compound is said to be water-immiscible if less than 3%, preferably less than 2%, for example less than 1% by weight, of this compound is in a form dissolved in water.

For the purposes of the invention, a fatty substance capable of dispersing in water, for example capable of generating micelles in water, is not considered to be water-miscible.

In the context of the invention, the term “nonaqueous medium” is intended to mean a medium that is significantly free of water, in particular comprising less than 10% by weight of water, for example less than 5% by weight of water, for example less than 3% by weight of water, preferably less than 1% by weight of water.

The nonaqueous medium according to the invention can in particular be used for preparing compositions in the fields of cosmetics, agrochemistry, pharmacy, the oil industry, the automobile industry, in the fields of inks, coatings, etc.

It should be understood that the nonaqueous medium according to the invention can represent all or part of the compositions of which it is intended to modify the properties, in particular the rheological properties.

According to a first variant, the composition may comprise only a nonaqueous medium according to the invention, and therefore be free of an aqueous phase. In this case, the nonaqueous medium according to the invention represents all of the composition of which it is intended to modify the properties, in particular the rheological properties.

According to a second variant, the composition may comprise, in addition to the nonaqueous medium according to the invention, an aqueous phase or a nonaqueous medium which is different in nature than the nonaqueous medium of which it is intended to modify the properties, in particular the rheological properties. This variant corresponds, for example, to compositions of (direct, inverse or multiple) emulsion type. In this case, the nonaqueous medium according to the invention represents only a part of the composition of which it is intended to modify the properties, in particular the rheological properties. Of course, according to this second variant, the modification of the properties, in particular the rheological properties, of the nonaqueous medium according to the invention can cause a modification of the properties, in particular the rheological properties, of all of the composition incorporating this nonaqueous medium. For example, when the composition of which it is intended to modify the properties, in particular the rheological properties, is in the form of a water-in-oil emulsion, the modification of the properties, in particular the rheological properties, of the nonaqueous medium (oil) can advantageously lead to a modification of the properties, in particular the rheological properties, of all of the emulsion, i.e. of the nonaqueous continuous medium, but also of the emulsified aqueous medium.

The invention thus relates to a copolymer of which the backbone is obtained by radical polymerization:
of a fatty substance (A) comprising unsaturations and/or hydroxyl functions; and
of at least one monomer (B) comprising at least one function that can be polymerized by radical polymerization and that comprises either at least one linear or branched alkyl chain, with said alkyl chain preferably comprising from 16 to 44 carbon atoms, more preferentially at least 18 carbon atoms, for example at least 20 carbon atoms, in particular 22 carbon atoms or 44 carbon atoms, or a reactive function.

Preferably, the copolymer is a copolymer of which the backbone is obtained by radical polymerization:
of a fatty substance (A) comprising unsaturations; and
of at least one monomer (B) comprising at least one function that can be polymerized by radical polymerization and that comprises at least one linear or branched alkyl chain comprising from 16 to 44 carbon atoms, more preferentially at least 18 carbon atoms, for example at least 20 carbon atoms, in particular 22 carbon atoms or 44 carbon atoms.

For the purposes of the invention, the reactive function may preferably be a function capable of reacting by means of a substitution reaction. By way of examples of such reactive functions, mention may, for example, be made of halogens, for example chlorine; alcohols; amines; acids; amides or else epoxies.

According to one embodiment, the monomer (B) can therefore comprise at least one function that can be polymerized by radical polymerization and one reactive function selected from halogens, for example chlorine; alcohols; amines; acids; amides and epoxies.

When the monomer (B) comprises at least one reactive function, for example as defined above, said function is then used for the grafting of at least one linear or branched alkyl chain, with said alkyl chain preferably comprising from 16 to 44 carbon atoms, more preferentially at least 18 carbon atoms, for example at least 20 carbon atoms, in particular 22 carbon atoms or 44 carbon atoms.

For example, it can involve a grafting reaction carried out by substitution between, on the one hand, a monomer (B) of vinylbenzyl halide type and a compound of fatty amine type capable of introducing into the structure of the copolymer an alkyl chain as defined previously.

It should be noted that, in the context of the present invention, the monomer (B) may comprise several, in particular 2, alkyl chains as defined previously.

For example, it is possible to use a monomer of dialkylacrylamide type, for example dioctylacrylamide, or else a dialkylstyrene.

In the context of the invention, the expression “between x and y” should be understood to include the values x and y. According to the invention, this expression also means from x to y.

According to the invention, the fatty substance (A) may be selected from mono, di and triglycerides of fatty acids and methyl or ethyl esters thereof, which are unmodified or modified (hydrogenation, hydroxylation, alcoxylation, alkylation, etc.); unsaturated hydrocarbons of which the carbon-based chain comprises at least one double or triple bond (for example: alkenes, alkynes and aromatic compounds) and/or which are hydroxylated; fatty acids of which the carbon-based chain comprises at least one double or triple bond (for example: alkenes, alkynes and aromatic compounds) and/or which are hydroxylated; fatty alcohols; fatty amines . . . etc., animal oils or oils of animal origin, preferably fish oils and in particular fish oils comprising omega-3 fatty acids, for example sardine oil, and derivatives thereof; silicone oils; terpene compounds; synthetic resins carrying a labile proton (hydroxyl, primary and secondary amine, thiol, etc., functions) and/or at least one unsaturation, for example resins based on polybutadiene (for example: the Krasol resins from Cary Valley) or on polypropylene (for example: the Trilene resins from Lion Copolymer), alcohols (for example: the Koresin phenolic resins from BASF), esters (for example: the USP/PE unsaturated polyester resins from Dow), ethers, nonaromatic or aromatic amides, and mixtures thereof.

According to one embodiment, the fatty substance (A) may be oleic acid.

The fatty substance (A) is preferably a vegetable oil or an oil of vegetable origin, for example selected from:
triglycerides of saturated or unsaturated fatty acids comprising at least 12 carbon atoms and preferably from 14 to 22 carbon atoms; they may be natural triglycerides, such as vegetable oils or oils of vegetable origin of the rapeseed oil, soybean oil, groundnut oil, butter oil, cottonseed oil, linseed oil, coconut oil, olive oil, palm oil, grapeseed oil, fish oil, castor oil or copra oil type;
esters of triglycerides of saturated or unsaturated fatty acids comprising at least 12 carbon atoms and preferably from 14 to 22 carbon atoms, in particular as defined previously, and especially the methyl and ethyl esters thereof;
   or an animal oil or oil of animal origin, for example a fish oil;
   or mixtures thereof.

Advantageously, the fatty substance (A) may be a vegetable oil or an oil of vegetable origin, selected, for example, from rapeseed oil, soybean oil, corn oil, castor oil, groundnut oil, butter oil, cottonseed oil, linseed oil, coconut oil, olive oil, palm oil, grapeseed oil, copra oil, and mixtures thereof. Rapeseed oil, in particular, is suitable for the invention, as are castor oil, corn oil and soybean oil.

According to one embodiment, the fatty substance (A) may be selected from castor oil and rapeseed oil.

Preferably, the fatty substance (A) is castor oil.

For the monomers (B) according to the invention, the expression “function that can be polymerized” is generally intended to mean any function capable of polymerizing via the radical process.

These functions are well known to those skilled in the art. By way of illustration, it may in particular be a function selected from acrylate, methacrylate, acrylamide, methacrylamide, vinyl, in particular allyl or vinyl ether, and styrene functions.

In particular, the monomer (B) may be selected from:
alkyl acrylates;
alkyl methacrylates;
alkylacrylamides;
alkylmethacrylamides;
alkyl vinyls, in particular alkyl allyls or alkyl vinyl ethers; and
alkylstyrenes;
   in which the alkyl is a linear or branched chain preferably comprising from 16 to 44 carbon atoms, more preferentially at least 18 carbon atoms, for example at least 20 carbon atoms, in particular 22 carbon atoms or 44 carbon atoms.

Preferably, the monomer (B) is selected from:
alkyl acrylates;
alkyl methacrylates;
alkylacrylamides;
alkylmethacrylamides;
   in which the alkyl is a linear or branched chain preferably comprising from 16 to 44 carbon atoms, more preferentially at least 18 carbon atoms, for example at least 20 carbon atoms, in particular 22 carbon atoms or 44 carbon atoms.

More preferentially, the monomer (B) is selected from:
alkyl acrylates;
alkyl methacrylates;
   in which the alkyl is a linear or branched chain preferably comprising from 16 to 44 carbon atoms, more preferentially at least 18 carbon atoms, for example at least 20 carbon atoms, in particular 22 carbon atoms or 44 carbon atoms.

Preferably, the monomer (B) may be selected from:
alkyl acrylates or methacrylates, wherein the alkyl is a linear or branched chain preferably comprising from 16 to 44 carbon atoms, more preferentially at least 18 carbon atoms, for example at least 20 carbon atoms, in particular 22 carbon atoms or 44 carbon atoms; and in particular from poly(ethoxylated and/or propoxylated) C₃-C₃₀, preferably C₁₆-C₃₀, more preferentially at least C₁₈, for example at least C₂₂, aliphatic alcohol acrylates or methacrylates, the aliphatic part of which is, as appropriate, substituted with one or more hydroxyl(s) preferably at the end of an aliphatic group;
alkylacrylamides or alkylmethacrylamides, in which the alkyl is a linear or branched chain preferably comprising from 16 to 44 carbon atoms, more preferentially at least 18 carbon atoms, for example at least 20 carbon atoms, in particular 22 carbon atoms or 44 carbon atoms; and in particular selected from poly(ethoxylated and/or propoxylated) C₃-C₃₀, preferably C₁₆-C₃₀, more preferentially at least C₁₈, for example at least C₂₂, aliphatic alcohol acrylamides or methacrylamides, the aliphatic part of which is, as appropriate, substituted with one or more hydroxyl(s) preferably at the end of an aliphatic group;
alkylstyrenes, in which the alkyl is a linear or branched chain preferably comprising from 16 to 44 carbon atoms, more preferentially at least 18 carbon atoms, for example at least 20 carbon atoms, in particular 22 carbon atoms or 44 carbon atoms, and derivatives thereof, for example comprising halogenated functions and/or hydroxylated functions and/or amine functions; preferably vinyl benzyl chloride, and the styrene comprising an alkyl chain preferably having from 16 to 44 carbon atoms, preferably located in the para-position;
alkyl vinyls, in which the alkyl is a linear or branched chain comprising in particular from 16 to 44 carbon atoms, preferably at least 18 carbon atoms, for example at least 20 carbon atoms; and in particular selected from allyl alkyl esters of which the alkyl chain at the end of the ester function comprises in particular from 16 to 44 carbon atoms, preferably at least 18 carbon atoms, for example at least 22 carbon atoms; or
mixtures thereof.

As indicated previously, the monomer (B) may comprise several, and in particular two, alkyl chains, with each of the alkyl chains being as defined above.

Particularly preferably, the monomer (B) is selected from an alkyl acrylate in which the alkyl chain comprises 22 carbon atoms, in particular behenyl acrylate, or an alkyl acrylate in which the alkyl chain comprises 44 carbon atoms.

Particularly preferably, the monomer (B) is behenyl acrylate.

According to the invention, at least one monomer (C) may also be used for preparing the copolymer according to the invention. The monomer (C) is selected from neutral C_{N} monomers; anionic or potentially anionic C_{A} monomers; cationic or potentially cationic C_{C} monomers; zwitterionic C_{Z} monomers; hydrophobic Cₚ monomers; and mixtures thereof.

The expression “anionic or potentially anionic monomers” is intended to mean monomers which comprise at least one anionic or potentially anionic group. Anionic groups are groups which exhibit at least one negative charge (generally associated with one or more cations, such as alkali metal or alkaline-earth metal, for example sodium, compound cations, or with one or more cationic compounds such as ammonium), regardless of the pH of the medium in which the copolymer is present. Potentially anionic groups are groups which may be neutral or may exhibit at least one negative charge depending on the pH of the medium in which the copolymer is present.

The expression “cationic or potentially cationic monomers” is intended to mean monomers which comprise at least one cationic or potentially cationic group. Cationic groups are groups which exhibit at least one positive charge (generally associated with one or more anions, such as the chloride ion, the bromide ion, a sulfate group, a methyl sulfate group), regardless of the pH of the medium in which the copolymer is present. Potentially cationic groups are groups which may be neutral or may exhibit at least one positive charge depending on the pH of the medium in which the copolymer is present.

The term “neutral groups” is intended to mean groups which do not exhibit a charge, regardless of the pH of the medium in which the copolymer is present.

The neutral C_{N} monomers may in particular be selected from the following monomers:
hydroxyalkyl esters of acids which are α,β-ethylenically unsaturated, preferably hydroxyethyl acrylate, hydroxyethyl methacrylate and hydroxypropyl acrylate;
α,β-ethylenically unsaturated amides, preferably acrylamide, methacrylamide, dimethylacrylamide and hydroxymethylacrylamide;
α,β-ethylenically unsaturated monomers comprising a water-soluble polyoxyalkylene segment with or without an alkyl chain, preferably polyethylene glycol acrylate or methacrylate, with or without alkyl chain, having a molecular weight between 350 and 5000 g/mol (it being understood that this value does not take into account the optional alkyl chain);
vinyl alcohol;
vinyl lactams;
ureido α,β-ethylenically unsaturated monomers, preferably (methacrylamidoethyl)-2-imidazolidinone;
vinylpyrrolidone;
mixtures thereof.

Preferably, the C_{N} monomers are selected from (meth)acrylamides, in particular acrylamide, methacrylamide, dimethylacrylamide and hydroxymethylacrylamide, vinylpyrrolidone, hydroxyethyl acrylate and polyethylene glycol methacrylate, with or without alkyl chain, having a molecular weight between 350 and 5 000 g/mol (it being understood that this value does not take into account the optional alkyl chain).

The anionic or potentially anionic C_{A} monomers may be selected from the following monomers:
monomers which have at least one carboxylic function, for example α,β-ethylenically unsaturated carboxylic acids, the corresponding anhydrides and water-soluble salts thereof, preferably acrylic acid or anhydride, methacrylic acid or anhydride, maleic acid or anhydride, fumaric acid, itaconic acid, N-methacryloylalanine, N-acryloylglycine, and water-soluble salts thereof;
monomers which have at least one sulfate or sulfonate function or one corresponding acid function, preferably 2-sulfooxyethyl methacrylate, vinylbenzenesulfonic acid, allylsulfonic acid, 2-acrylamido-2-methylpropanesulfonic acid, sulfoethyl acrylate or methacrylate, or sulfopropyl acrylate or methacrylate, and water-soluble salts thereof;
monomers which have at least one phosphonate or phosphate function or one corresponding acid function, preferably vinylphosphonic acid, ethylenically unsaturated phosphate esters;
mixtures thereof.

The C_{A} monomers that are particularly preferred are acrylic acid and methacrylic acid.

The cationic or potentially cationic C_{C} monomers may be selected from:
monomers with a secondary, tertiary or quaternary amine function, preferably propyldimethylamine methacrylamide, 4-vinylaniline and diallyldimethylammonium chloride (DADMAC);
mixtures thereof.

The zwitterionic C_{Z} monomers, i.e. monomers comprising two opposite charges, may be selected from:
alkylsulfonates or carboxylates or phosphonates of dialkylammonioalkyl acrylates or methacrylates, -acrylamides or methacrylamides;
heterocyclic betaine monomers;
dialkylammonioalkylallyl alkylsulfonates or carboxylates or phosphonates;
phosphobetaines of formulae:

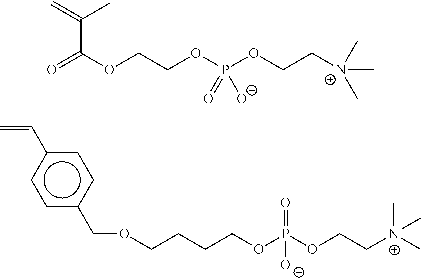

betaines derived from cyclic acetals.

Particularly preferably, C_{Z} monomers are selected from dimethyl(methacrylamidopropyl)(3-sulfopropyl)ammonium betaine (SPP) and 1-(3-sulfopropyl)-2-vinylpyridinium betaine (SPV).

The Cₚ monomers are selected from:
alkyl acrylates;
   alkyl methacrylates;
   alkylacrylamides;
   alkylmethacrylamides;
   alkyl vinyls, in particular alkyl allyls or alkyl vinyl ethers; and
   alkylstyrenes;
      in which the alkyl is a linear or branched chain preferably comprising from 1 to 44 carbon atoms, more preferentially from 1 to 22 carbon atoms.

Preferably, the Cp monomers are selected from:
alkyl acrylates or methacrylates, in which the alkyl is a linear or branched chain preferably comprising from 1 to 44 carbon atoms, preferably from 1 to 22 carbon atoms; and in particular from poly(ethoxylated and/or propoxylated) C₃-C₃₀, preferably C₁₆-C₃₀, more preferentially at least C₁₈, for example at least C₂₂, aliphatic alcohol acrylates or methacrylates, the aliphatic part of which is, as appropriate, substituted with one or more hydroxyl(s) preferably at the end of an aliphatic group;
alkylacrylamides or alkylmethacrylamides, in which the alkyl is a linear or branched chain preferably comprising from 1 to 44 carbon atoms, more preferentially from 1 to 22 carbon atoms; and in particular selected from poly(ethoxylated and/or propoxylated) C₃-C₃₀, preferably C₁₆-C₃₀, more preferentially at least C₁₈, for example at least C₂₂, aliphatic alcohol acrylamides or methacrylamides, the aliphatic part of which is, as appropriate, substituted with one or more hydroxyl(s) preferably at the end of an aliphatic group;
alkylstyrenes, in which the alkyl is a linear or branched chain preferably comprising from 1 to 44 carbon atoms, more preferentially from 1 to 22 carbon atoms, and derivatives thereof, for example comprising halogenated functions and/or hydroxyl functions and/or amine functions; preferably vinyl benzyl chloride, and the styrene comprising an alkyl chain preferably having from 1 to 44 carbon atoms, preferably located in the para-position;
alkyl vinyls, in which the alkyl is a linear or branched chain comprising in particular from 1 to 44 carbon atoms, preferably from 1 to 22 carbon atoms; and in particular selected from allyl alkyl esters of which the alkyl chain at the end of the ester function comprises in particular from 1 to 44 carbon atoms, preferably from 1 to 22 carbon atoms;
mixtures thereof.

The Cp monomers that are preferred are 2-ethylhexyl acrylate and styrene.

According to the invention, the degree of grafting of the fatty substance (A) in the copolymer may reach 100%, for example 80%, and preferably between 5% and 60%. The degree of grafting is calculated according to one of the following formulae (eq1), (eq2) or (eq3) depending on whether the fatty substance (A) comprises, respectively, only unsaturations (eq1), only hydroxyl functions (eq2) or both unsaturations and hydroxyl functions (eq3):

$$\begin{matrix}degree of grafting = ( \frac{A 2}{A 1} ) & ( eq 1 ) \\ degree of grafting = ( \frac{B 2}{B 1} ) & ( eq 2 ) \\ degree of grafting = ( \frac{A 2}{A 1} ) + ( \frac{B 2}{B 1} ) & ( eq 3 )\end{matrix}$$

in which:

$$( \frac{A 2}{A 1} ) = ( \frac{number of unsaturations polymerized}{number of initial unsaturations} )$$

$$( \frac{B 2}{B 1} ) = ( \frac{number of hydroxyl functions substituted}{number of initial hydroxyl functions} )$$

Of course, the degree of grafting can vary according to the nature of the fatty substance, and in particular according to the number of unsaturations and/or of hydroxyl functions present in the fatty substance.

In particular:
when the fatty substance is castor oil, the degree of grafting of the fatty substance can be between 5% and 60%, for example between 5% and 40%, for example between 10% and 30%, in particular between 10% and 15%;
when the fatty substance is rapeseed oil, the degree of grafting of the fatty substance can be between 5% and 80%, for example between 10% and 60%, for example between 15% and 50%;
when the fatty substance is oleic acid, the degree of grafting of the fatty substance can be between 5% and 80%, for example between 10% and 70%, for example between 15% and 60%.

The numbers of unsaturations and of hydroxyl functions are generally known and depend on the fatty substance under consideration; they can also be calculated by NMR.

With regard to the number of unsaturations polymerized and the number of hydroxyl functions substituted, they can also be calculated by NMR.

In particular, these calculations can be carried out by proton NMR, using deuterated chloroform CDCl₃ as solvent.

The copolymer of the invention advantageously has a molecular weight between 10 000 and 1×10⁶ g/mol, preferably between 15 000 and 500 000 g/mol, more preferentially between 20 000 and 250 000 g/mol, for example less than 200 000 g/mol, for example less than 150 000 g/mol, for example less than 100 000 g/mol, for example between 15 000 and 80 000 g/mol, for example between 20 000 and 80 000 g/mol, for example between 25 000 and 80 000 g/mol.

These molecular weights can be calculated in particular by gas chromatography (GC) with polystyrene calibration, with THF under hot conditions.

According to one preferred embodiment, the invention relates to a copolymer of which the backbone is obtained by radical polymerization:
of a fatty substance (A) comprising unsaturations, selected from vegetable oils or oils of vegetable origin as described previously, in particular castor oil;
of at least one monomer (B) comprising at least one function that can be polymerized by radical polymerization and that comprises at least one linear or branched alkyl chain comprising from 16 to 44 carbon atoms, in particular behenyl acrylate;
   with the degree of grafting of said fatty substance being between 10% and 30%, in particular between 10% and 15%; and
   with the molecular weight of said copolymer being less than 150 000 g/mol, for example between 25 000 and 80 000 g/mol.

Surprisingly, it has been shown by the inventors that it is possible, by virtue of the copolymer according to the invention, to modify the properties, in particular the rheology, of a nonaqueous medium with polymers having a molecular weight much lower than that of the polymers normally used to modify the properties, in particular the rheology, of such nonaqueous media.

According to one embodiment, the copolymer of the invention can be obtained by radical polymerization of a fatty substance (A), of at least one monomer (B) and optionally of at least one monomer (C) as defined previously, in proportions such that:
the molar amount of said monomer (B) relative to said monomer (C) ranges from 10% to 100%, for example from 25% to 100%,
the molar amount of said monomer (B) relative to said fatty substance (A) ranges from 1% to 99%, for example from 10% to 99%,
the molar amount of the mixture of said monomer (B) and of said monomer (C) relative to said fatty substance (A) ranges from 1% to 99%, for example from 10% to 99%.

The copolymer according to the invention may be in liquid form or in solid form.

The copolymer according to the invention has modified properties, in particular modified rheological properties, compared with the fatty substance (A), in particular the presence of a rheological threshold and/or modification of the viscosity, and/or modifications of the gelling properties.

Advantageously, the copolymer of the invention exhibits a rheological threshold which is identifiable on the curve representing the shear rate as a function of the stress applied. Thus, the value of the shear rate remains zero (or substantially zero) until application of a minimum stress, denoted “threshold stress value”. Without wishing to be bound to a particular theory, the studies carried out by the inventors in the context of the invention make it possible to put forward that it is at least partly this particular rheological behavior that will make it possible to keep compounds, for example solid or liquid active compounds, for example phytosanitary active agents, in suspension, in dispersions using the copolymer of the invention.

In the context of the invention, the threshold stress value, below which the shear rate remains zero or substantially zero, is generally at least 0.01 Pa, preferably at least 0.1 Pa, for example at least 0.5 Pa, in a wide temperature range, in particular both at ambient temperature, in particular from 10 to 30° C., and at higher temperatures, for example between 35 and 60° C., in particular at 45 and 54° C., and over time, in particular both for at least 3 days, and preferably for at least 7 days, for example for at least 15 days.

More particularly, in the context of the invention, the threshold stress value below which the shear rate remains zero or substantially zero, may be at least 0.01 Pa, preferably at least 0.1 Pa, for example at least 0.5 Pa under standard accelerated aging conditions, for example at least 15 days at 54° C. or at least 8 weeks at 45° C.

The thresholds can be determined by means of creep tests on an AR2000ex rheometer (TA Instruments) in particular at 25 and 54° C. A cone-plate geometry is used with an aluminum cone with an angle of 1 deg 59 min 2 sec, a diameter of 60 mm and a truncation of 57 μm.

A pre-shear of 10 s⁻¹ for 10 s is carried out and the sample is then left to stand for 2 min. Successive stresses of 1.4, 1.6, 1.8, 2, 2.3, 2.5, 2.8 and 3 Pa at 25° C. and of 0.1, 0.2, 0.3, 0.4, 0.5, 0.6, 0.7, 0.8, 1, 1.2 and 1.5 Pa at 54° C. are each applied for 2 min. During this time, the deformation of the fluid which results therefrom is measured. Recovery tests lasting 6 min are carried out at the end of each stress applied. The results obtained make it possible to plot a curve of the stress as a function of the shear rate. The shear rate for each stress applied is then evaluated by taking the slope of the straight line between 80 and 120 s.

The threshold stress corresponds to the intersection of the two characteristic straight lines of the break of the slope.

Advantageously, the copolymer of the invention has a modified viscosity, in particular a higher viscosity, compared with the fatty substance (A).

Advantageously, the copolymer of the invention has modified gelling and/or viscosity properties compared with the fatty substance (A), and in particular a higher viscosity and/or a better gelling ability.

The invention also relates to a process (P1) for preparing a copolymer by radical polymerization of a fatty substance (A) comprising unsaturations and/or hydroxyl functions with at least one monomer (B) comprising at least one function that can be polymerized by radical polymerization and that comprises either at least one linear or branched alkyl chain, with said alkyl chain preferably comprising from 16 to 44 carbon atoms, more preferentially at least 18 carbon atoms, for example at least 20 carbon atoms, in particular 22 carbon atoms or 44 carbon atoms, or a reactive function.

For the process (P1) of the invention, the monomers (B) and the fatty substance (A) are as defined for the copolymer according to the invention.

In addition, according to the invention, at least one monomer (C) selected from neutral C_{N} monomers; anionic or potentially anionic C_{A} monomers; cationic or potentially cationic C_{C} monomers; zwitterionic C_{Z} monomers; hydrophobic Cₚ monomers; and mixtures and combinations thereof, can be used for preparing the copolymer.

The monomers (C) are as defined for the copolymer according to the invention.

According to the invention, the process (P1) is advantageously carried out at a temperature of between 50 and 150° C., preferably between 60 and 120° C.

According to the invention, the radical polymerization is carried out in the presence of a radical initiator which can be selected from the radicals known to those skilled in the art. By way of example, mention may be made of peroxides and azo compounds, preferably lauryl peroxide and tert-butyl peroxybenzoate. It can in particular be used in a molar amount ranging from 0.1% to 50%, for example from 1% to 20%, relative to the monomer (B).

Preferably, for the process (P1) of the invention, the B/A molar ratio corresponding to the monomer (B)/fatty substance (A) molar ratio is between 0.1/9.9 and 9.9/0.1, preferably between 2/8 and 9/1.

Preferably, for the process (P1) of the invention, the molar amount of the monomer (C) relative to the monomer (B) ranges from 0 to 90%, preferably from 0 to 75%.

The implementation of the process (P1) according to the invention makes it possible to modify the properties, in particular the rheological properties, of the fatty substance (A). It advantageously makes it possible to provide a rheological threshold and/or to modify the viscosity (in particular to increase the viscosity) and/or to modify the gelling properties.

The present invention also relates to a process (P2) for modifying the properties, in particular the rheological properties, of a nonaqueous medium by addition to this medium of at least one copolymer according to the invention.

The nonaqueous medium according to the invention can in particular be used for preparing compositions in the fields of cosmetics, agrochemistry, pharmacy, the oil industry, the automobile industry, in the fields of inks, coatings, etc.

It should be understood that the nonaqueous medium according to the invention can represent, for example, all or part of the oily phase of an emulsion, in particular of an inverse or multiple emulsion.

The nonaqueous medium can in particular be used in agrochemical formulations, for example, for preparing oil dispersions (also abbreviated to “ODs”), inverse emulsions; in solvents such as diesters and derivatives thereof, DMSO, alcohols and derivatives thereof, ethers and derivatives thereof; compositions resulting from the oil industry, such as in the field of aromatic petroleum oils, oil-based drilling fluids; in cosmetic compositions, for example in inverse emulsions, creams, lipsticks, deodorants, varnishes; in industrial compositions such as lubricating compositions, coating compositions, paints, stripping compositions, inks, greases; in pharmaceutical compositions, in particular in drug-release supports, in compositions of active agents in suspension in an oil, etc.

Preferably, the nonaqueous medium can be used for preparing agrochemical compositions such as oily dispersions or inverse or multiple emulsions.

According to the invention, the nonaqueous medium may also comprise supplementary agents, in particular selected from:
emulsifiers;
thickeners;
active agents, in particular phytosanitary, cosmetic or pharmaceutical active agents, preferably phytosanitary agents;
water-soluble polymers;
polysaccharides, in particular guar gum, xanthan gum, etc.; or
formulating agents.

The addition of the copolymer according to the invention to the nonaqueous medium makes it possible to modify the properties, in particular the rheological properties, of said medium, in particular to provide a rheological threshold and/or to modify its viscosity (in particular to increase its viscosity) and/or to modify its gelling properties.

The addition of the copolymer according to the invention makes it possible in particular to provide a rheological threshold that will make it possible to prepare stable compositions comprising solid particles in suspension, said stability being with respect to the sedimentation of the solid particles.

In the context of the invention, the expression “stable composition comprising solid particles in suspension” is intended to mean a composition which exhibits substantially no sedimentation and/or phase separation of the dispersed compound, in a wide temperature range, in particular both at ambient temperature, in particular between 10 and 30° C., and at higher temperatures, for example between 35 and 60° C., in particular at 45 and 54° C., in particular at least 16 days at ambient temperature and at least 15 days at 54° C.

More particularly, in the context of the invention, “a stable composition comprising solid particles in suspension” is a composition which exhibits substantially no sedimentation and/or phase separation of the dispersed compound under standard accelerated aging conditions, for example at least 15 days at 54° C. or at least 8 weeks at 45° C.

The addition of the copolymer according to the invention may also make it possible to modify the viscosity of the starting nonaqueous medium (in particular to increase its viscosity), in particular for limiting smears or running during the use of the compositions, in particular of the cosmetic products.

The addition of the copolymer according to the invention may also make it possible to modify the gelling properties of the nonaqueous medium, which can in particular prove to be useful for preparing compositions which are solid but easy to spread, for example in the context of a cosmetic composition such as sticks.

Depending on the rheological properties that it is desired to obtain, those skilled in the art are able to determine the amount of copolymer to be added to the nonaqueous medium. Preferably, in the process (P2), i.e. when the copolymer is present as an additive in the nonaqueous medium, the copolymer is added in a proportion of between 0.1% and 20% by weight, for example between 0.5% and 15% by weight, preferably between 1% and 7% by weight relative to the total weight of the nonaqueous medium.

According to another embodiment variant, the copolymer according to the invention may be present as main agent in the nonaqueous medium of which it is intended to modify the properties, in particular the rheological properties, and in particular the copolymer may represent more than 40% by weight, for example more than 50% by weight, for example more than 60% by weight, for example more than 70% by weight, for example more than 80% by weight, for example more than 90% by weight, or may even represent 100% by weight of said nonaqueous medium.

The present invention also relates to a composition constituted totally or partially of the copolymer according to the invention.

Thus, the invention relates to a composition constituted totally of a copolymer according to the invention. In this case, the composition may be used as an additive to be added to a nonaqueous medium so as to modify the properties thereof, in particular the rheological properties thereof. It is not out of the question for a minor fraction of the composition to be constituted of other substances, in particular active agents, emulsifiers, formulating agents, water-soluble polymers, polysaccharides, in particular guar gum, xanthan gum, with the exception of the nonaqueous media, without these other substances modifying or disrupting the properties, in particular the rheological properties, of this composition.

The invention also relates to a composition comprising at least one copolymer according to the invention as a mixture within a nonaqueous medium. Such a composition has modified rheological properties compared with those of the nonaqueous medium, thus allowing the use of the composition as a formulation base in particular for preparing compositions in the fields of cosmetics, agrochemistry, pharmacy, the oil industry, the automobile industry, in the field of inks, coatings, etc.; in particular for preparing agrochemical compositions, for example for preparing oil dispersions, inverse emulsions; in solvents such as diesters and derivatives thereof, DMSO, alcohols and derivatives thereof, ethers and derivatives thereof; compositions resulting from the oil industry, such as in the field of aromatic petroleum oils, oil-based drilling fluids; in cosmetic compositions, for example in inverse emulsions, creams, lipsticks, deodorants, varnishes; in industrial compositions such as lubricating compositions, coating compositions, paints, stripping compositions, inks, greases; in pharmaceutical compositions, in particular in drug-release supports, in compositions of active agents in suspension in an oil, etc.

The rheological properties of the nonaqueous medium which are modified by the copolymer will make it possible to prepare dispersions of solid compounds over time and in a wide temperature range. In particular, the dispersion may be stable after storage for 14 days at 54° C. and even after storage for 8 weeks at 45° C.

The composition may also comprise other substances, such as, in particular:
active agents, for example phytosanitary, pharmaceutical or cosmetic active agents, preferably phytosanitary active agents;
emulsifiers;
thickeners;
water-soluble polymers;
polysaccharides, in particular guar gum, xanthan gum, etc.; and/or
formulating agents.

More particularly, the present invention relates to compositions that are emulsifiable by mixing with water, comprising:
a nonpolar medium;
a compound, for example a phytosanitary active agent, dispersed within said nonpolar medium;
a copolymer according to the invention; and
an emulsifier.

In the context of the present invention, the expression “compositions that are emulsifiable by mixing with water” is intended to mean compositions which, after mixing with water, make it possible to obtain emulsions.

The emulsifiable composition preferably contains little or no water. Thus, the emulsifiable composition of the invention comprises from 0 to 5% of water, preferably from 0 to 1% of water, for example from 0 to 0.1% of water, by weight relative to the total weight of the composition.

The emulsifiable composition according to the invention may be any type of emulsifiable composition which, in particular according to the nature of the compound dispersed, can have various uses. The emulsifiable composition according to the invention may, for example, be a cosmetic composition, an agrochemical composition, a pharmaceutical composition, a composition that can be used in the oil, automobile, ink, coating, etc., industries. More particularly, the emulsifiable composition according to the invention is an agrochemical composition, in particular an oil dispersion (OD).

In the context of the invention, a compound is said to be dispersed within a nonpolar medium when less than 5% by weight, preferably less than 3% by weight, for example less than 1% by weight, of this compound is in a form dissolved in said nonpolar medium, in particular at ambient temperature, generally from 15 to 30° C., or even at a temperature ranging up to 54° C.

The dispersed compound may be a dispersed solid compound or a dispersed liquid compound.

According to one embodiment of the invention, the dispersed compound is a dispersed solid compound. In the context of the invention, the term “solid compound” is intended to mean a compound of which the melting point is greater than or equal to 50° C.

According to another embodiment of the invention, the dispersed compound is a dispersed liquid compound. In the context of the invention, the term “liquid compound” is intended to mean a compound of which the melting point is below 50° C.

In the context of the present invention, the inventors have now demonstrated that the compositions comprising the abovementioned compounds prove to be stable, in particular at ambient temperature and at higher temperatures, in particular at 45° C., or even up to 54° C. The compositions according to the invention also prove to be stable over time, in particular at least 16 days at ambient temperature and at least 15 days at 54° C. In particular, advantageously, no sedimentation and/or phase separation are observed at these temperatures and for these periods.

More concretely, the studies that have been carried out by the inventors have now made it possible to demonstrate that the combination of the nonpolar medium and of the copolymer according to the invention make it possible to induce a viscoelastic behavior of this nonpolar dispersant medium which proves to be sufficient to allow the compound to be maintained in suspension and the formulation to flow, both at ambient temperature, in particular between 10 and 30° C., in particular at least 16 days at ambient temperature, and at a higher temperature, for example between 35 and 60° C., in particular at 45 and 54° C., in particular under standard accelerated aging conditions (for example at least 15 days at 54° C. or at least 8 weeks at 45° C.), despite the presence of the emulsifier.

The use of the combination of the nonpolar medium and the copolymer according to the invention advantageously makes it possible to generate a rheological threshold in the curve representing the shear as a function of the stress applied. Thus, the value of the shear remains zero (or substantially zero) until application of a minimum stress, denoted “threshold stress value”. Without wishing to be bound to a particular theory, the studies carried out by the inventors in the context of the invention make it possible to put forward that it is at least partly this particular rheological behavior that makes it possible to keep the compound, in particular the phytosanitary active agents, in suspension, in the compositions of the invention. In the context of the invention, the threshold stress value, below which the shear remains zero or substantially zero, is generally greater than 0.2 Pa, preferably between 0.2 and 1 Pa, in a wide temperature range, in particular both at ambient temperature, in particular between 10 and 30° C., and at higher temperatures, for example between 35 and 60° C., in particular at 45 and 54° C., in particular at least 16 days at ambient temperature, and in particular under standard accelerated aging conditions (for example at least 15 days at 54° C. or at least 8 weeks at 45° C.). Surprisingly, it turns out that this surprising rheological behavior is obtained in the presence of the emulsifier and over a wide temperature range. The presence of the emulsifier makes it possible to prepare a relatively stable emulsion from the composition by mixing it with water and over a wide temperature range. Thus, the compositions of the invention prove to be particularly suitable as ODs, allowing compositions, in particular phytosanitary active agents, to be maintained in suspension, within a nonpolar medium, in particular an oil, which allows them to be stored and transported in concentrated form and allows them to be easily converted into an emulsion by simple mixing with water on the site where they are used.

In the context of the invention, the term “stable composition” is intended to mean a composition which exhibits substantially no sedimentation and/or phase separation of the dispersed compound, in a wide temperature range, in particular both at ambient temperature, in particular between 10 and 30° C., and at higher temperatures, for example between 35 and 60° C., in particular at 45 at 54° C., in particular at least 16 days at ambient temperature and at least 15 days at 54° C. Preferably, a stable composition is a composition which exhibits substantially no sedimentation and phase separation of the dispersed compound, in a wide temperature range, in particular both at ambient temperature, in particular between 10 and 30° C., and at higher temperatures, for example between 35 and 60° C., in particular at 45 and 54° C., in particular at least 16 days at ambient temperature and in particular under standard accelerated aging conditions (for example at least 15 days at 54° C. or at least 8 weeks at 45° C.).

Preferably, a composition according to the invention remains stable after storage for 15 days at 54° C. Likewise, a composition according to the invention can remain stable after storage for 8 weeks at 45° C.

### Dispersed Compound

The compound, in particular phytosanitary active agent, present in the dispersed state within the composition according to the invention, can be present in a high concentration, which can, in certain cases, range up to 30%, for example 40%, or even 50% by weight relative to the total weight of the composition, without loss of the stability of the dispersion.

However, most commonly, the concentration of compound present in the dispersed state (generally dispersed phytosanitary active agent) is from 0.1% to 20% by weight, for example from 0.5% to 10% by weight, for example from 1% to 8% by weight relative to the total weight of the composition.

The compound present in the dispersed state within the composition according to the invention may be selected from any compound which is insoluble in the nonpolar medium of this composition. In the context of the invention, a compound is said to be insoluble when less than 5%, preferably less than 3%, for example less than 1% of this compound is in a form dissolved in the nonpolar medium. It may typically be a phytosanitary active agent, namely an active agent suitable for improving the growth of plants, for treating or preventing plant diseases, or for combating parasites or pests likely to inhibit or modify the growth of the plant. A phytosanitary active agent may, for example, be a pesticide, such as an insecticide, a bactericide, a fungicide, a herbicide, or a plant growth regulator or harmful organism growth regulator, or mixtures thereof.

As fungicidal active agent, mention may be made of nucleic acid synthesis inhibitors; mitosis and cell division inhibitors; respiratory inhibitors; compounds capable of acting as an uncoupler; ATP production synthesis inhibitors; amino acid or protein biosynthesis inhibitors; signal transduction inhibitors; inhibitors of lipid synthesis at the membrane; ergosterol biosynthesis inhibitors; cell wall synthesis inhibitors; melanin biosynthesis inhibitors; compounds capable of triggering plant defense mechanisms; compounds capable of acting at several sites; or mixtures thereof.

As insecticidal active material, mention may be made of acetylcholinesterase (AChE) inhibitors; GABA-dependent chloride pathway antagonist compounds; nicotinic acetylcholine receptor antagonist compounds; allosteric acetylcholine receptor-modulating compounds; chloride pathway-activating compounds; compounds of which the mode of action is unknown or is not specific, for example gassing agents; clothes moth growth inhibitors; oxidative phosphorylation inhibitors; ATP disruptors; oxidative phosphorylation-uncoupling compounds; microbial disruptors of the insect digestive membrane; chitin biosynthesis inhibitors; molting disruptors; ecdysone disruptors; anti-octopaminergics; electron transport (site II or site III) inhibitors; electron transport (site I) inhibitors; fatty acid biosynthesis inhibitors; neuronal inhibitors for which the mode of action has not been elucidated; compounds capable of modifying ryanodine receptors; or mixtures thereof.

By way of preference of phytosanitary active agents that can be used in the emulsifiable compositions, mention may in particular be made, in a nonlimiting manner, of sulfonylureas such as bensulfuron-methyl, chlorimuron-ethyl, chlorsulfuron, metsulfuron-methyl, nicosulfuron, sulfomethuron-methyl, triasulfuron, tribenuron-methyl, azoles such as difenconazole, the triazole fungicide family, such as azaconazole, bromuconazole, cyproconazole, difenoconazole, diniconazole, epoxyconazole, fenbuconazole, flusilazole, myclobutanyl, tebuconazole, alternatively ametryn, diuron, linuron, novaluron, chlortoluron, isoproturon, metamitron, diazinon, aclonifen, atrazine, chlorothalonil, bromoxynil, bromoxynil heptanoate, bromoxynil octanoate, mancozeb, manebe, zineb, phenmedipham, propanyl, the phenoxyphenoxy series, the heteroaryloxyphenoxy series, CMPP, MCPA, 2,4-D, simazine, the active products of the imidazolinone series, the organophosphorus compound family, with in particular azinphos-ethyl, azinphos-methyl, alachlor, chlorpyriphos, diclofop-methyl, fenoxaprop-p-ethyl, methoxychlor, cypermethrin, alpha-cypermethrin, phenmedipham, propanil, oxyfluorfen, dimethoate, imidacloprid, propoxur, benomyl, deltamethrin, fenvalerate, abamectin, amicarbazone, bifenthrin, carbosulfan, cyfluthrin, ethofenprox, fenoxaprop-ethyl, fluazifop-p-butyl, flufenoxuron, hexazinone, lambda-cyalothrin, permethrin, prochloraz, methomyl, fenoxycarb, cymoxanil, chlorothalonyl, neonicotinoid insecticides, triadimefon, triadimenol, strobilurins such as pyraclostrobin, picoxystrobin, azoxystrobin, famoxadone, kresoxym-methyl and trifloxystrobin; or mixtures thereof.

The invention in particular proves to be suitable for the use of active agents of the sulfonylurea family, such as nicosulfuron, and azoles such as tebuconazole.

The composition according to the invention may also comprise, according to one particular embodiment, one or more other active compounds (generally other phytosanitary active agents or compounds which modulate the phytotoxicity of active agents (safeners)) which are dissolved or miscible in the nonpolar medium. The compositions of this type, which comprise active agents in combination in two distinct forms, are formulations that are very suitable in particular in agrochemistry, and which are sometimes referred to as “combos”. According to one embodiment, the dispersed compound may be a solid compound dispersed within the compositions of the invention (generally a phytosanitary active agent or a mixture of phytosanitary active agents in the solid state). It is preferably in the form of dispersed objects (particles or particle aggregates) having sizes of less than 50 μm, in particular less than 20 μm, advantageously between 1 and 15 μm, for example of the order of 10 μm or less. The size of these objects in suspension can be determined according to any means known per se, for example by optical microscopy or light scattering.

According to another embodiment, the dispersed compound may be a liquid compound dispersed within the compositions of the invention (generally a phytosanitary active agent or a mixture of phytosanitary active agents in the liquid state). It is preferably in the form of droplets having sizes of less than 5 μm, in particular between 0.5 and 5 μm, for example between 1 and 3 μm. The size of these droplets can be determined by any means known per se, for example by optical microscopy or light scattering.

Particularly preferably, the dispersed compound is nicosulfuron.

### Nonpolar Medium

For the purposes of the invention, the term “nonpolar medium” is intended to mean any constituent which is liquid at the temperature in which the composition is prepared or used, and which, lying in the Hansen solubility space (Handbook of solubility parameters and other cohesion parameters—Allan F. M. BARTON, CRC Press Inc., 1983—), exhibits the following parameters:

δP of Keesom interactions less than 10 (J/cm³)^{1/2}

δH of hydrogen bonds less than 10 (J/cm³)^{1/2}

δD of London interactions greater than 15 (J/cm³)^{1/2}

By way of example of a nonpolar medium or nonpolar dispersion medium, mention may be made of:
triglycerides of saturated or unsaturated fatty acids comprising at least 12 carbon atoms and preferably from 14 to 22 carbon atoms; they may be synthetic triglycerides or preferably natural triglycerides, such as vegetable oils or oils of vegetable origin of the rapeseed oil, soybean oil, groundnut oil, butter oil, cottonseed oil, linseed oil, coconut oil, olive oil, palm oil, grapeseed oil, castor oil or copra oil type, or animal oils or oils of animal origin, for example fish oils, in particular fish oils comprising omega-3 fatty acids;
esters of triglycerides of saturated or unsaturated fatty acids comprising at least 12 carbon atoms and preferably from 14 to 22 carbon atoms (in particular the methyl and ethyl esters thereof);
aromatic petroleum fractions;
aromatic solvents (anisole, toluene, for example);
terpene compounds (D-limonene, L-limonene, for example);
mixtures of dimethyl succinate/adipate/glutarate diesters;
aliphatic hydrocarbons comprising at least 6 carbon atoms (isooctane, kerosene, gasoline, diesel, mineral oils (in particular liquid paraffin), lubricating oils, etc.); or
mixtures thereof.

Advantageously, the nonpolar medium present in the composition of the invention comprises, or even is, a mixture of triglycerides, for example a vegetable oil or oil of vegetable origin, selected, for example, from rapeseed oil, soybean oil, corn oil, castor oil, groundnut oil, butter oil, cottonseed oil, linseed oil, coconut oil, olive oil, palm oil, grapeseed oil, copra oil, and mixtures thereof. Rapeseed oil, in particular, is suitable for the invention, as are corn oil and soybean oil, and mixtures thereof.

The nonpolar medium is preferably rapeseed oil.

The preferential use of vegetable oil or oil of vegetable origin advantageously makes it possible to dispense with the solvents generally used, such as xylene, naphthalene, N-methylpyrrolidone, cyclohexanone or alternatively isophorone, in emulsifiable concentrates (ECs), and which have a not insignificant environmental impact.

Use may also be made of other natural oils, such as, for example, animal oils or oils of animal origin, in particular fish oils, for example fish oils comprising omega-3 fatty acids.

The nonpolar medium present in the composition may also comprise, or even be, an aromatic petroleum fraction such as the Solvesso products (for example, Solvesso 100 which is a mixture of C₉ to C₁₀ dialkyl and trialkylbenzenes, Solvesso 150 which contains as main mixture C₁₀ to C₁₁ alkylbenzenes, or Solvesso 200, which contains mainly C₁₀ to C₁₄ alkyl naphthalenes), preferably Solvesso 200 ND, preferably as a mixture with other compounds, such as esters of vegetable oils or oils of vegetable origin, in particular rapeseed oil, of the Phytorob 926-25 type sold by the company Novance or Amesolve CME available from Ametech. This embodiment is in particular suitable for compositions comprising compounds which modulate the phytotoxicity of active agents, of safener type.

The nonpolar medium may be identical in nature to or different in nature than the fatty substance (A) according to the invention.

According to one embodiment, the nonpolar medium and the fatty substance (A) may be vegetable oils or oils of vegetable origin. For example, the nonpolar medium may be rapeseed oil and the fatty substance (A) may be castor oil.

Generally, whatever the nature of the nonpolar medium, for example of the oil, the latter may be present in the composition according to the invention at a content ranging from 40% to 99% by weight, preferably from 40% to 90%, this content preferably being at least 60% by weight, for example at least 65% by weight, for example between 65% and 75% by weight, relative to the total weight of the composition.

It is not out of the question to mix aromatic fractions with vegetable oils or oils of vegetable or natural origin, in variable proportions. The use of such aromatic fractions can in particular be implemented when the compositions comprise, in addition to the dispersed active agent, a safener, it being possible for these petroleum fractions to then advantageously make it possible to dissolve the safener.

For example, the nonpolar medium may comprise vegetable or natural oils and aromatic fractions in proportions by weight ranging from 20:80 to 80:20, preferably ranging from 65:35 to 75:25.

According to one embodiment, the nonpolar medium may comprise vegetable or natural oils and aromatic fractions in proportions by weight ranging from 40:60 to 60:40 and for example 50:50.

It is also not out of the question to mix methyl esters with vegetable oils or oils of vegetable or natural origin, in variable proportions.

Whatever the nature of the copolymer according to the invention used, it is preferably employed at a concentration that is sufficiently low to avoid any solidification of the medium, but that is nevertheless sufficient to induce the desired rheological modification of the medium so as to provide stabilization of the compounds in the dispersed state in the composition.

The amount of copolymer according to the invention in the emulsifiable composition is generally between 0.5% and 40% by weight, for example between 1% and 20% by weight, preferably between 2% and 10% by weight, for example between 3% and 8% by weight, relative to the total weight of the composition.

To this effect, in particular when the nonpolar medium present in the composition comprises at least, for example comprises predominantly, for example comprises at least 50% by weight of, or even is, a triglyceride-based oil, such as a vegetable oil for example, the concentration of copolymer according to the invention is preferably less than 10%, more advantageously less than 8% by weight relative to the total weight of the composition, typically between 1% and 8%, for example between 3% and 6%, in particular of the order of from 4% to 6% by weight relative to the total weight of the composition.

Particularly preferably, in the emulsifiable compositions according to the invention, the nonpolar medium is a vegetable oil or oil of vegetable origin as described previously.

In particular, when the copolymer according to the invention is a copolymer of which the backbone is obtained by radical polymerization:
of a fatty substance (A) comprising unsaturations, selected from vegetable oils or oils of vegetable origin as described previously, in particular castor oil;
of at least one monomer (B) comprising at least one function that can be polymerized by radical polymerization and that comprises at least one linear or branched alkyl chain comprising from 16 to 44 carbon atoms, in particular behenyl acrylate;
   with the degree of grafting of said fatty substance being between 10% and 30%, in particular between 10% and 15%; and
   with the molecular weight of said copolymer being less than 150 000 g/mol, for example between 25 000 and 80 000 g/mol,
   it may be advantageously formulated with a vegetable oil or oil of vegetable origin as defined previously, in particular with rapeseed oil.

This embodiment proves in particular to be suitable for the use of active agents of the sulfonylurea family, such as nicosulfuron, and azoles such as tebuconazole.

### Emulsifier

The emulsifier, present in the composition according to the invention, is suitable for emulsifying the nonpolar medium, for example the oil, during mixing of the composition with water or an aqueous medium; it may be selected from surfactants suitable for emulsifying the specific nonpolar medium, for example the specific oil, present in the emulsifiable composition.

Thus, for example when the nonpolar medium comprises at least, for example comprises predominantly, for example comprises at least 50% by weight of, or even is, a mixture of triglycerides such as a vegetable oil, the emulsifier may be selected from nonionic surfactants of the type such as fatty acids or esters, for instance the esters, glycol esters glycerol esters, PEG esters, PEG esters of fatty acids, sorbitol esters, sorbitol esters which are ethoxylated, acids which are ethoxylated, or ethoxypropoxylated, esters and triglycerides (Alkamuls® family from Rhodia), in particular ethoxylated castor oils, and mixtures thereof.

The emulsifier may in particular be selected from ethoxylated castor oils, polyethylene glycol esters of fatty acids, and sorbitan esters, and mixtures thereof.

By way of example of ethoxylated castor oils that are suitable for the invention, mention may in particular be made of the products Alkamuls® OR 36, Alkamuls® RC, Alkamuls® R81, Alkamuls® 696 and Alkamuls OR/10 available from the company Rhodia.

By way of example of polyethylene glycol esters of fatty acids that are suitable for the invention, mention may in particular be made of the product Alkamuls VO/2003, available from the company Rhodia.

By way of example of sorbitan esters that are suitable for the invention, mention may in particular be made of the products Alkamuls T/85-V and Alkamuls T/80 available from the company Rhodia.

Emulsifiers that are particularly suitable, in particular when the nonpolar medium comprises at least, for example comprises predominantly, for example comprises at least 50% by weight of, or even is, a mixture of triglycerides such as a vegetable oil, are polyethylene glycol esters of fatty acids, alone or in combination with another surfactant.

An emulsifier which is particularly suitable in the context of the emulsifiable compositions of the invention is Alkamuls VO/2003, available from the company Rhodia.

For other nonpolar media, in particular nonpolar media comprising at least, for example comprising predominantly, for example comprising at least 50% by weight of, or even being, aromatic petroleum fractions and/or aliphatic hydrocarbons comprising at least 6 carbon atoms, suitable emulsifiers are in particular anionic surfactants such as sulfonates, aliphatic sulfonates, sulfonates carrying ester or amide groups, such as isothioanates (sulfoesters), taurates (sulfoamides) sulfosuccinates, sulfosuccinamates, or else sulfonates not carrying amide ou ester groups, such as alkyl diphenyl oxide disulfonate, alkyl naphthalene sulfonate, naphthalene/formaldehyde sulfonates with, for example, dodecyl benzene sulfonates (Rhodacal® family from Rhodia, for instance Rhodacal® 60 BE), alone or in combination with:
one or more nonionic surfactants of the type such as fatty acids or esters, for instance the esters, glycol esters, glycerol esters, PEG esters, sorbitol esters, sorbitol esters which are ethoxylated, acids which are ethoxylated, or ethoxypropoxylated, esters and triglycerides (Alkamuls® family from Rhodia), in particular ethoxylated castor oils; preferably one or more nonionic surfactants of ethoxylated castor oil type, for instance those sold by the company Rhodia under the references Alkamuls® OR 36, Alkamuls® RC, Alkamuls® R81 and Alkamuls® 696, and/or
one or more surfactants selected from compounds based on styrylphenol such as distyrylphenol or tristyrylphenol, which may be ethoxylated or ethoxypropoxylated, phosphated or sulfated, for example the Soprophor® family sold by the company Rhodia, for instance Soprophor® DSS7, Soprophor® BSU, Soprophor® 3D33, Soprophor® 4D384 or Soprophor® 796P.

The surfactants of Rhodasurf®, Rhodacal® and Alkamuls® type available from the company Rhodia are in particular suitable.

A combination of surfactants that is suitable for these nonpolar media is the product sold under the name Geronol MOE/02-K by the company Rhodia.

The content of emulsifier may vary according in particular to the nature of the nonpolar medium and to the nature of the emulsifier used.

Generally, the amount of emulsifier within the emulsifiable composition according to the invention is typically between 3% and 30% by weight relative to the total weight of the emulsifiable composition.

Preferably, when the nonpolar medium comprises at least, for example comprises predominantly, for example comprises at least 50% by weight of, or even is, a mixture of triglycerides such as a vegetable oil, the content of emulsifier is preferentially between 5% and 25% by weight, for example between 10% and 20% by weight, for example approximately 15% by weight, relative to the total weight of the emulsifiable composition. Preferably, when the nonpolar medium comprises at least, for example comprises predominantly, for example comprises at least 50% by weight of, or even is, an aliphatic hydrocarbon comprising at least 6 carbon atoms, for example a mineral oil, the content of emulsifier is preferentially between 3% and 15% by weight, for example between 4% and 10% by weight, for example approximately 5% by weight, relative to the total weight of the emulsifiable composition.

In one particular embodiment, in the emulsifiable composition according to the invention, the nonpolar medium is a vegetable oil or a mixture of vegetable oils, as described above, and the emulsifier is a polyethylene glycol ester of fatty acids (in particular of Alkamuls VO/2003 type, available from the company Rhodia).

In another particular embodiment, in the emulsifiable composition according to the invention, the nonpolar medium is a vegetable oil or a mixture of vegetable oils, as described above, the emulsifier is a polyethylene glycol ester of fatty acids (in particular of Alkamuls VO/2003 type, available from the company Rhodia) and the dispersed compound is nicosulfuron.

The nonpolar medium may in particular be rapeseed oil and the emulsifier a polyethylene glycol ester of fatty acids (in particular of Alkamuls VO/2003 type, available from the company Rhodia).

This embodiment is particularly suitable when the copolymer according to the invention is a copolymer of which the backbone is obtained by radical polymerization:
of a fatty substance (A) comprising unsaturations, selected from vegetable oils or oils of vegetable origin as described previously, in particular castor oil;
of at least one monomer (B) comprising at least one function that can be polymerized by radical polymerization and that comprises at least one linear or branched alkyl chain comprising from 16 to 44 carbon atoms, in particular behenyl acrylate; with the degree of grafting of said fatty substance being between 10% and 30%, in particular between 10% and 15%; and
   with the molecular weight of said copolymer being less than 150 000 g/mol, for example between 25 000 and 80 000 g/mol.

### Optional Ingredients

The emulsifiable composition according to the invention may also optionally comprise other ingredients, in particular selected from:
fillers, for example particles of silica, such as, for example, the silica sold by the company Evonik under the reference Aerosil® 200;
salts, in particular carbonates or sulfates, for instance sodium carbonate or ammonium sulfonate (the addition of such salts may in particular be recommended when hard water is used);
antifoams, such as, for example, the product sold by the company Rhodia under the reference Silcolapse RG22;
and mixtures thereof.

Generally, the content of fillers (if present), for example silica particles, is less than 1% by weight, preferably less than 0.5% by weight, preferably less than 0.2% by weight, for example between 0.05% and 0.1% by weight, relative to the total weight of the emulsifiable composition according to the invention.

Generally, the content of salts (if present), for example of sodium carbonate or of ammonium sulfonate, is less than 1% by weight, preferably less than 0.5% by weight, preferably less than 0.2% by weight, for example between 0.05% and 0.1% by weight, relative to the total weight of the emulsifiable composition according to the invention.

Generally, the content of antifoam (if present), is less than 1% by weight, preferably less than 0.5% by weight, preferably less than 0.2% by weight, for example between 0.05% and 0.1% by weight, relative to the total weight of the emulsifiable composition according to the invention.

Depending on its composition, the emulsifiable composition according to the invention can typically be prepared by carrying out a process (P1) comprising the following steps:

(i) the nonpolar medium and the emulsifier are mixed (these compounds preferably being introduced in this order to form the mixture), said mixture is advantageously sheared, in particular by means of a device of deflocculator blade type; then
(ii) the copolymer according to the invention is added, the medium obtained is advantageously sheared, in particular by means of a device of deflocculator blade type; heating is carried out (if necessary) until the copolymer has dissolved (generally between 60° C. and 75° C.), before cooling the whole mixture with stirring; then
(iii) the compound to be dispersed is added, and the medium obtained is advantageously sheared, in particular by means of a device of Ultraturrax type.

According to one embodiment, at the end of step (iii), the composition can be subjected to milling, in particular in a wet mill.

An additional milling step may in particular be advantageous for reducing the size of the particles when the compound to be dispersed is a solid compound of considerable size, in order to obtain, for example, a size of the particles or aggregates as a dispersion of preferably less than 20 μm, more advantageously less than 10 μm.

An additional milling step may also be advantageous for other reasons, when the compound to be dispersed is a liquid compound or a solid compound of suitable size. It may in particular make it possible to improve the mixing of the ingredients.

The emulsifiable compositions according to the invention may also be obtained according to a process (P2), comprising the following steps:

(a) the nonpolar medium, the emulsifier and the compound to be dispersed are mixed (these compounds preferably being introduced in this order to form the mixture), said mixture is advantageously sheared, in particular by means of a device of Ultraturrax type; then
(b) the copolymer according to the invention, dissolved in the nonpolar medium, is added while hot (generally between 60° C. and 75° C.).

According to one embodiment, step (b) is preceded by a step (b1) of milling the mixture obtained in step (a), in particular in a wet mill, preferably, in the case where the compound to be dispersed is a solid composition, until a size of the particles or aggregates as a dispersion of less than 20 μm, more advantageously less than 10 μm, is obtained. In this embodiment, step (b) is also carried out during the milling.

An additional milling step may also be advantageous for other reasons, when the compound to be dispersed is a liquid compound or a solid compound of suitable size. It may in particular make it possible to improve the mixing of the ingredients.

The emulsifiable compositions according to the invention may also be obtained according to a process (P3), comprising the following steps:

(I) the nonpolar medium and the compound to be dispersed are mixed (these compounds preferably being introduced in this order to form the mixture), said mixture is advantageously sheared, in particular by means of a device of Ultraturrax type; then
(II) the copolymer according to the invention, dissolved in the nonpolar medium, and the emulsifier are added while hot (generally between 60° C. and 75° C.).

According to one embodiment, step (II) is preceded by a step (II1) of milling the mixture obtained in step (I), in particular in a wet mill, preferably, in the case where the compound to be dispersed is a solid composition, until a size of the particles or aggregates as a dispersion of less than 20 μm, more advantageously less than 10 μm, is obtained. In this embodiment, step (II) is also carried out during the milling.

An additional milling step may also be advantageous for other reasons, when the compound to be dispersed is a liquid compound or a solid compound of suitable size. It may in particular make it possible to improve the mixing of the ingredients.

The composition according to the invention can in particular be used for the on-site preparation of emulsions for the delivery of compounds, in particular solid compounds, in particular of phytosanitary active agents, by dilution in water. The emulsion obtained may, for example, be used for spraying onto the aerial parts, in particular the foliage, of plants.

In this context, the composition according to the invention may be used for forming emulsions of oil-in-water type comprising the compound in the dispersed state. The formation of such an emulsion is generally carried out by mixing an emulsifiable composition (of OD type) according to the invention with an aqueous phase, in particular with water. Preferably, the composition according to the invention/water weight ratio is between 0.1:100 and 10:100, more preferentially between 1:100 and 5:100, for example between 2:100 and 3:100.

The emulsions obtained from the compositions according to the invention allow, in particular, good dispersion of the active agents at the surface of the plants to be treated. Furthermore, the use of the active agent within a nonpolar vehicle generally allows good compatiblization of the active agent with the surface to be treated, which can in particular allow persistence of the activity on the surface of the plant, better penetrability and good staying power of the active agent, including during bad weather (wind and rain in particular). The emulsions obtained exhibit in particular better bioavailability and increased resistance to being washed away.

In the case where the copolymer according to the invention has a high viscosity, the inventors have, moreover, discovered that the addition of a particular liquid emulsifier can, particularly advantageously, result in a liquid, homogeneous, bifunctional (rheological agent and emulsifier) concentrated mixture which is flowable and therefore easier to handle. This bifunctional mixture is particularly advantageous in the case in particular of OD formulations, which require the presence of such an emulsifier in the final formulation.

Thus, according to another embodiment, the present invention also relates to concentrated compositions comprising a copolymer according to the invention and an emulsifier. Preferably, the concentrated composition according to the invention consists of a mixture of a copolymer according to the invention and an emulsifier.

Preferably, the emulsifier is selected from those required in the final formulation, for example in the final formulation of OD type. Particularly preferably, the emulsifier is a polyethylene glycol ester of fatty acids. It may in particular be the product Alkamuls VO/2003, available from the company Rhodia.

According to one embodiment, the invention relates to a concentrated composition consisting of a mixture of a copolymer according to the invention and an emulsifier, in a copolymer according to the invention/emulsifier weight ratio of between 10:90 and 40:60, preferably between 15:85 and 35:65, for example of the order of 20:80.

According to another embodiment, this concentrated composition may also comprise additional fillers, for example silica particles, in a content of less than 5% by weight, for example less than 2% by weight, for example less than 1% by weight, for example between 0.1% and 0.5% by weight, relative to the total weight of the composition.

By way of additional fillers that are particularly suitable for this embodiment, mention may in particular be made of the silica sold by the company Evonik under the reference Aerosil® 200.

According to another embodiment, the invention also relates to a liquid concentrate based on a copolymer according to the invention and on a vegetable oil or oil of vegetable origin, with said copolymer according to the invention and said vegetable oil or oil of vegetable origin being present in a copolymer according to the invention/vegetable oil or oil of vegetable origin weight ratio of between 3:97 and 40:60, in particular between 5:95 and 20:80, for example of the order of 10:90.

By way of vegetable oil or oil of vegetable origin that is particularly suitable for this embodiment, mention may in particular be made of rapeseed oil.

The present invention will now be described using nonlimiting examples.

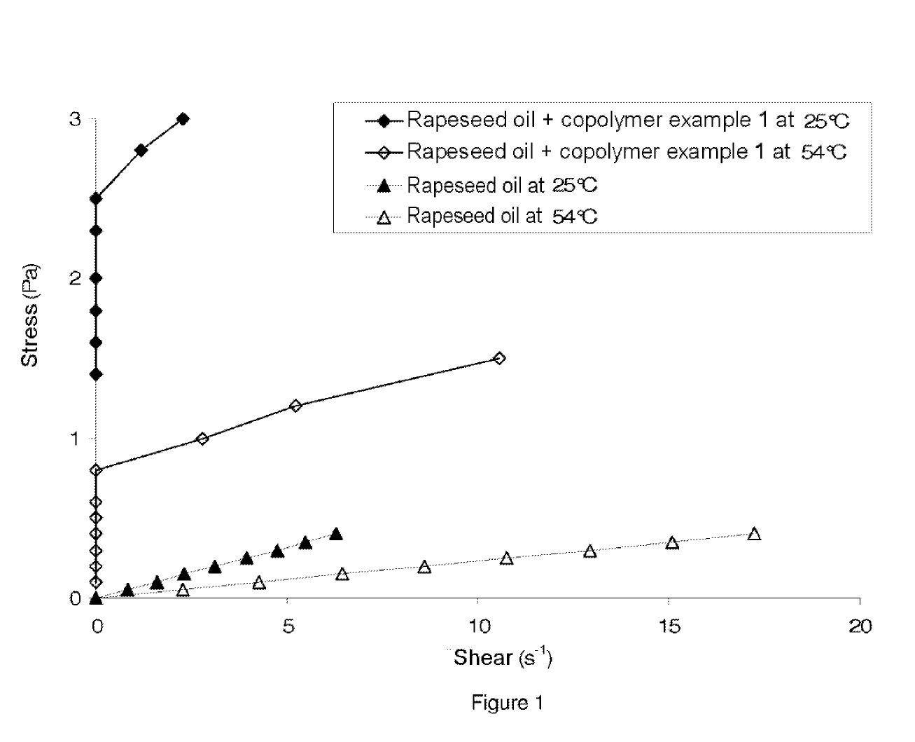

FIG. 1 represents the change in stress as a function of the shear rate of a rapeseed oil at 25 and 54° C. and of a composition of rapeseed oil and of a copolymer according to the invention at 25 and 54° C.

### EXAMPLE 1

### Preparation of a Copolymer According to the Invention

20 g of C₁₈-C₂₂ alkyl acrylate (behenyl acrylate at 70%) and 16.4 g of castor oil are introduced into a 100 ml three-necked flask equipped with a condenser, a mechanical stirrer (anchor), a nitrogen bubbler and a heating oil bath. The whole assembly is heated to 80° C. and then the temperature is maintained for 4 h. At 80° C., 1.29 g of lauryl peroxide are added. After 2 h at 80° C., 0.65 g of lauryl peroxide is added.

The characteristics of the copolymerized fatty substance obtained are the following:
degree of grafting of the castor oil: 12.4%;
degree of conversion into poly(behenyl acrylate): 100%;
Mw (measured by relative GC with polystyrene calibration): 66 150 g/mol.

### EXAMPLE 2

### Preparation of a Copolymer According to the Invention

20 g of C₁₈-C₂₂ alkyl acrylate (behenyl acrylate at 70%), 21.4 g of rapeseed oil and 1.29 g of lauryl peroxide are introduced into a 100 ml three-necked flask equipped with a condenser, a mechanical stirrer (anchor), a nitrogen bubbler and a heating oil bath. The whole assembly is heated at 90° C. for 4 h.

The characteristics of the copolymerized fatty substance obtained are the following:
degree of grafting of the rapeseed oil: 42.3%;
degree of conversion into poly(behenyl acrylate): 100%;
Mw (measured by relative GC with polystyrene calibration): 16 230 g/mol.

### EXAMPLE 3

### Preparation of a Copolymer According to the Invention

20 g of C₁₈-C₂₂ alkyl acrylate (behenyl acrylate at 70%) and 6.88 g of oleic acid are introduced into a 100 ml three-necked flask equipped with a condenser, a mechanical stirrer (anchor), a nitrogen bubbler and a heating oil bath. The whole assembly is heated to 80° C. and then the temperature is maintained for 4 h. At 80° C., 1.29 g of lauryl peroxide are added. After 2 h at 80° C., 0.65 g of lauryl peroxide is added.

The characteristics of the copolymerized fatty substance obtained are the following:
degree of grafting of the oleic acid: 50.5%;
degree of conversion into poly(behenyl acrylate): 98.5%
Mw (measured by relative GC with polystyrene calibration): 517 000 g/mol.

### EXAMPLE 4

### Preparation of a Copolymer not in Accordance with the Invention (Counterexample)

154.41 g of dodecyl acrylate and 254.93 g of castor oil are introduced into a 1 l glass reactor equipped with a condenser, a mechanical stirrer (anchor), a nitrogen bubbler and a jacket in which a heat-transfer fluid circulates. The whole assembly is heated to 60° C. and maintained for 1 h. At 60° C., 7.24 g of lauryl peroxide are added. The medium is then heated to 70° C. and maintained for 2 h, then heated at 75° C. for 2 h and then at 80° C. for 4 h. After 1 h at 80° C., 3.62 g of lauryl peroxide are added.

The characteristics of the copolymerized fatty substance obtained are the following:
degree of grafting of the castor oil: 6.4%;
degree of conversion into poly(lauryl acrylate): 98%
Mw (measured by relative GC with polystyrene calibration): 282 000 g/mol.

### EXAMPLE 5

### Evaluation of the Rheology of the Copolymer of Example 1

### Sample Preparation:

1.1 g of the copolymer of example 1 (i.e. 5.5% by weight) are weighed into a 50 ml flask which has a 25 mm×8 mm magnetic bar, and then 18.9 g of rapeseed oil (i.e. 94.5% by weight) are added. The previous mixture is heated and is maintained with stirring at 300 rpm. The copolymer of example 1 is dissolved at a temperature of 60±2° C. The solution obtained is then clear and yellowish. Said solution is then cooled to ambient temperature with stirring at 300 rpm. The previously clear solution thickens. The sample is then stored at ambient temperature before the rheological measurements are carried out.

### Measurement of the Threshold by Creep Test

The determination of the thresholds at 25 and 54° C. of the mixture is carried out using creep tests on an AR2000ex rheometer (TA Instruments). A cone-plate geometry is used with an aluminum cone with an angle of 1 deg 59 min 2 sec, a diameter of 60 mm and a truncation of 57 μm.

For each of the two temperatures, a pre-shear of 10 s⁻¹ for 10 s is carried out and the sample is then left to stand for 2 minutes. Succesive stresses of 1.4, 1.6, 1.8, 2, 2.3, 2.5, 2.8 and 3 Pa at 25° C. and of 0.1, 0.2, 0.3, 0.4, 0.5, 0.6, 0.7, 0.8, 1, 1.2 and 1.5 Pa at 54° C. are each applied for 2 minutes. During this time, the deformation of the fluid which results therefrom is measured. Recovery tests lasting 6 min are carried out at the end of each stress applied. The shear rate for each stress applied is then obtained by taking the slope of the straight line between 80 and 120 s.

The curve giving the stress versus the shear rate is then plotted (FIG. 1). The threshold stress, when there is one, corresponds to the intersection of the two characteristic straight lines of the break of the slope and is reported in table 1 at the temperatures of 25° C. and 54° C.

Additionally, viscosity measurements at shear rates of 1, 10 and 100 s⁻¹ were also carried out with the same cone as previously, thereby making it possible to evaluate the increase in the viscosity of the system at various shears and its flowability at various temperatures.

The results are reconciled in the following table 1:

TABLE 1

| | Temperature 25° C. | | | | Temperature 54° C. | | | |
|---|---|---|---|---|---|---|---|---|
| Shear value | 1 s⁻¹ | 10 s⁻¹ | 100 s⁻¹ | Threshold | 1 s⁻¹ | 10 s⁻¹ | 100 s⁻¹ | Threshold |
| System | Viscosity (Pa · s) | | | (Pa) | Viscosity (Pa · s) | | | (Pa) |
| Rapeseed oil | 0.06 | 0.06 | 0.06 | 0 | 0.02 | 0.02 | 0.02 | 0 |
| Rapeseed oil with | 5.70 | 0.97 | 0.36 | 2.5 | 0.71 | 0.15 | 0.08 | 0.8 |
| 5.5% of the | | | | | | | | |
| copolymer of | | | | | | | | |
| example 1 added | | | | | | | | |

Rapeseed oil alone exhibits no threshold, either at 25° C. or at 54° C., and exhibits a constant viscosity at 1, 10 and 100 s⁻¹ (Newtonian behavior). The addition of the copolymer of example 1 to the rapeseed oil in an amount of 5.5% by weight makes it possible to obtain a considerable threshold of the order of 2.5 Pa at 25° C. and especially shows the presence of a considerable threshold of 0.8 Pa at the temperature of 54° C. The presence of such thresholds over this wide temperature range advantageously makes it possible, in dispersions of solid compounds in oil, to maintain solid active agents in suspension and also to maintain the stability of the composition. Furthermore, the addition of the copolymer of example 1 shows a significant increase in the viscosity of the system at various shear gradients, without, however, preventing the “flowability” of the system, thus confirming the pseudoplastic behavior of the copolymer according to the invention, which is an advantage in the preparation of various compositions, in particular cosmetic compositions, or alternatively with regard to coatings.

### EXAMPLE 6

### Evaluation of the Stability of a Complete Formulation Prepared with a Copolymer in Accordance with the Invention

6.6 g of the copolymer of example 1 are added, with a spatula, to a 250 ml Pyrex beaker, as are 24 g of emulsifier (polyethylene glycol esters of fatty acids sold by the company Rhodia under the name Alkamuls VO/2003) and 84.36 g of rapeseed oil, with a pipette. This mixture is then heated with stirring at 300 rpm until complete dissolution of the copolymer of example 1, and then cooled to ambient temperature with the same stirring. 5.04 g of nicosulfuron (solid active agent) are subsequently added, followed by homogenization with a spatula. The mixture is then subjected to 3 minutes in an Ultra Turrax T50 with a dual effect blade of D=45 mm, at 6000 rpm, before being subjected to wet milling in a MiniMotor Mill of the Eiger Torrance LTD brand, with a 50 ml capacity.

38 ml of SEPR ER 120 A, ZrO₂/SiO₂ beads 0.8 to 1.25 mm in diameter are used for the milling, which lasts 10 min at 3300 rpm (graduation 7).

It is verified under an optical microscope that all the particles are less than 10 μm in size.

TABLE 2

| | Content as % by |
|---|---|
| Formulation | weight |
| Nicosulfuron | 4.20 |
| Copolymer of example 1 | 5.50 |
| Alkamuls VO2003 | 20.00 |
| Rapeseed oil | 70.30 |

Various “standard” trade tests for agrochemical formulations are carried out on this formulation according to table 2 in order to be sure of the quality and the stability of said formulation.

Particularly of interest is the stability of the dispersion in oil (phase separation: syneresis and/or sedimentation) during aging tests (ambient temperature, −10° C. for 7 days, 54° C. for 15 days) and also its viscosity. Also of interest is the behavior of the dispersion in oil during its switching in water (pH, wet sieve residue, foam and stability of the emulsion generated at various times (0, 0.5 h, 24 h and redispersion at 24 h followed by wait of 0.5 h)).

The results are presented in table 3:

TABLE 3

| | | | Composition of table 2 | | |
|---|---|---|---|---|---|
| | | | Storage | | |
| | | | | 20 days at | |
| | | | ambient | | |
| | | | temperature | 7 days at −10° C. | 15 days at 54° C. |
| Syneresis | | | 0% | 0% | 5.0% |
| Flowability | | | Flowable | Difficult | Difficult flowability |
| | | | | flowability at | at 54° C. |
| | | | | −10° C. | Slightly yellow |
| Viscosity | Brookfield Viscosity (20 rpm) | | 1540(25° C.) | 1860(24° C.) | 2500(24° C.) |
| (mPa s⁻¹) | | | | | |
| pH (at 5%) | pH | | 6.9 | 6.9 | 6.9 |
| Wet sieve | Retention on 80 μm | | 0% | 0% | 0% |
| residue | sieve for 5% of | | | | |
| (inspiration | composition diluted in | | | | |
| taken from MT | mains water after | | | | |
| 185) | magnetic stirring | | | | |
| Foam (inspiration | Use of a 100 ml | | 0 immediate | 0 immediate | 0 immediate |
| taken from MT 47.2) | graduated cylinder, | | | | |
| | addition of 2 ml of | | | | |
| | composition to mains | | | | |
| | water, 30 inversions | | | | |
| | and observation of the | | | | |
| | amount of foam | | | | |
| Dispersion stability (inspiration taken from MT 180) | Waiting time before | | | | |
| Formulation diluted to | characterization | | | | |
| 2%, at 30 +/− 2° C., in mains water. | 0 h | Appearance of | White | White | Very slightly yellow |
| 100 ml graduated cyclinder used, inversion 10 times, | | the emulsion | emulsion. A | emulsion. A | emulsion. Some fatty |
| then observation after: | | | little fat stuck | little fat stuck to | objects stuck to the |
| | | | to the surface | the surface | surface |
| | 0.5 h | Cream (mm) | traces | 0.5 | 0.5 |
| | | Oil (mm) | 0 | 0 | 0 |
| | | Sediment (mm) | 0 | 0 | 0 |
| | 24 h | Cream (mm) | traces | 0.5 | 0.5 |
| | | Oil (mm) | 0 | 0 | 0 |
| | | Sediment (mm) | 0 | 0 | 0 |
| | | Redispersion | A little fat | A little fat stuck | Some fatty objects |
| | | | stuck to the | to the surface. | stuck to the surface. |
| | | | surface. | | |
| | 24.5 h | Cream (mm) | 0.5 | 0.5 | 0.5 |
| | | Oil (mm) | 0 | 0 | 0 |
| | | Sediment (mm) | 0 | 0 | 0 |

The results show a formulation that is completely stable at ambient temperature after 20 days and at −10° C. after 7 days since no phase separation appears (nor syneresis, nor sedimentation). The accelerated aging test (15 days at 54° C.) shows a small amount of syneresis (5%), and slight traces of oil at the bottom are observed. The formulation is flowable at ambient temperature and exhibits difficult flowability at −10° C. and 54° C., even after a return to ambient temperature.

A pH of 6.9 is measured after dilution of 1% by weight of the composition in mains water. The wet sieve residue test is good, since no residue is retained on the sieve. The foam test is excellent, since no residual foam is present after 30 inversions of the graduated cylinder.

The dispersion stability test is good, with a stable emulsion being obtained, since no trace of surface oil, nor of sediment, is present at the various observation times indicated, and slight creaming of at most 0.5 ml.

This cream is partially redispersible.

These results show that the use of a copolymer according to the invention makes it possible to prepare compositions comprising a dispersion of phytosanitary active agents that are stable over time and at temperature.

Of course, these results can not limit the invention to active agents of this type.

These results demonstrate in fact, more generally, that the copolymers of the invention make it possible to prepare compositions of all types (agrochemical, cosmetic, pharmaceutical, etc.) comprising at least one active agent, which are stable over time and over a wide temperature range.

### EXAMPLE 7

### Comparative Example Demonstrating the Impact of the Alkyl Chain Lengths on Performance Levels

Measurements of Threshold and Viscosity Using a Copolymer not in Accordance with the Invention (Counterexample 4)

### Sample Preparation:

1.1 g of the copolymer of counterexample 4 (i.e. 5.5% by weight) are weighed into a 50 ml flask which has a 25 mm×8 mm magnetic bar, and then 18.9 g of rapeseed oil (i.e. 94.5% by weight) are added. The previous mixture is heated and is maintained with stirring at 300 rpm. The copolymer of counterexample 4 is dissolved at a temperature of 60±2° C. The solution obtained is then clear and yellowish. Said solution is then cooled to ambient temperature with stirring at 300 rpm The sample is then stored at ambient temperature before the rheological measurements are carried out.

### Measurement of the Threshold by Creep Test

The determination of the thresholds at 25 and 54° C. of the mixture is carried out using creep tests on an AR2000ex rheometer (TA Instruments). A cone-plate geometry is used with an aluminum cone with an angle of 1 deg 59 min 2 sec, a diameter of 60 mm and a truncation of 57 μm.

For each of the two temperatures, a pre-shear of 10 s⁻¹ for 10 s is carried out and the sample is then left to stand for 2 minutes. Succesive stresses of 1.4, 1.6, 1.8, 2, 2.3, 2.5, 2.8 and 3 Pa at 25° C. and of 0.1, 0.2, 0.3, 0.4, 0.5, 0.6, 0.7, 0.8, 1, 1.2 and 1.5 Pa at 54° C. are each applied for 2 minutes. During this time, the deformation of the fluid which results therefrom is measured. Recovery tests lasting 6 min are carried out at the end of each stress applied. The shear rate for each stress applied is then obtained by taking the slope of the straight line between 80 and 120 s.

The curve giving the stress versus the shear rate is then plotted. The threshold stress, when there is one, corresponds to the intersection of the two characteristic straight lines of the break of the slope and is reported in table 4 at the temperatures of 25° C. and 54° C.

Additionally, viscosity measurements at shear rates of 1, 10 and 100 s⁻¹ were also carried out with the same cone as previously, thereby making it possible to evaluate the increase in the viscosity of the system at various shears and its flowability at various temperatures. The results are reconciled in the following table 4:

TABLE 4

| | Temperature 25° C. | | | | Temperature 54° C. | | | |
|---|---|---|---|---|---|---|---|---|
| Shear value | 1 s⁻¹ | 10 s⁻¹ | 100 s⁻¹ | Threshold | 1 s⁻¹ | 10 s⁻¹ | 100 s⁻¹ | Threshold |
| System | Viscosity (Pa · s) | | | (Pa) | Viscosity (Pa · s) | | | (Pa) |
| Rapeseed oil | 0.06 | 0.06 | 0.06 | 0 | 0.02 | 0.02 | 0.02 | 0 |
| Rapeseed oil with | 0.075 | 0.075 | 0.075 | 0 | 0.028 | 0.028 | 0.028 | 0 |
| 5.5% of the | | | | | | | | |
| copolymer of | | | | | | | | |
| counterexample 4 | | | | | | | | |
| added | | | | | | | | |

Rapeseed oil alone exhibits no threshold, either at 25° C. or at 54° C., and exhibits a constant viscosity at 1, 10 and 100 s⁻¹ (Newtonian behavior). The addition of the copolymer of counterexample 4 to the rapeseed oil in an amount of 5.5% by weight does not make it possible to significantly change the characteristics of the oil, since the viscosity remains similar and the behavior is still Newtonian, while the threshold is zero at 25° C. and at 54° C. The absence of thresholds over this wide temperature range does not make it possible, in dispersions of solid compounds in oil, to maintain solid active agents in suspension, as illustrated below.

Evaluation of the Stability of a Complete Formulation Prepared with a Copolymer not in Accordance with the Invention (Counterexample 4)

6.6 g of a copolymer not in accordance with the invention (counterexample 4) are added, with a spatula, to a 250 ml Pyrex beaker, as are 24 g of emulsifier (polyethylene glycol esters of fatty acids sold by the company Rhodia under the name Alkamuls VO2003) and 84.36 g of rapeseed oil, with a pipette. This mixture is then heated with stirring at 300 rpm until complete dissolution of the copolymer, and then cooled to ambient temperature with the same stirring. 5.04 g of nicosulfuron (solid active agent) are subsequently added, followed by homogenization with a spatula. The mixture is then subjected to 3 minutes in an Ultra Turrax T50 with a dual effect blade of D=45 mm, at 6000 rpm, before being subjected to wet milling in a MiniMotor Mill of the Eiger Torrance LTD brand, with a 50 ml capacity. 38 ml of SEPR ER 120 A, ZrO₂/SiO₂ beads 0.8 to 1.25 mm in diameter are used for the milling, which lasts 10 min at 3300 rpm (graduation 7).

It is verified under an optical microscope that all the particles are less than 10 μm in size.

TABLE 5

| | Content as % by |
|---|---|
| Formulation | weight |
| Nicosulfuron | 4.20 |
| Copolymer of | 5.50 |
| counterexample 4 | |
| Alkamuls VO2003 | 20.00 |
| Rapeseed oil | 70.30 |

Various “standard” trade tests for agrochemical formulations are carried out on this formulation according to table 6 in order to measure the stability of said formulation. Particularly of interest is the stability of the dispersion in oil (phase separation: syneresis and/or sedimentation) during aging tests (ambient temperature, 0° C. for 7 days, 54° C. for 15 days) and also its viscosity. The results are presented in table 6:

TABLE 6

| | 14 days at | | |
|---|---|---|---|
| | ambient | | |
| Storage | temperature | 7 days at 0° C. | 15 days at 54° C. |
| Syneresis | 68% | 56% | 68% |
| (phase separation) | | | |

The results show a formulation which is completely unstable at all temperatures, since the active agent (Nicosulfuron) sediments, leaving behind a phase separation of more than 50%. No supplementary characterization test was carried out because this formulation is not viable.

### EXAMPLE 8

### Evaluation of the Stability of a Complete Formulation Prepared with a Copolymer in Accordance with the Invention

3.908 g of the copolymer of example 1 are added, with a spatula, to a 250 ml Pyrex beaker, as are 26.022 g of emulsifier (polyethylene glycol esters of fatty acids sold by the company Rhodia under the name Alkamuls VO2003) and 91.926 g of rapeseed oil, with a pipette. 0.061 g of Aerosil 200 silica (Evonik) and also 0.121 g of sodium carbonate are added with a spatula. This mixture is then heated with stirring at 300 rpm until complete dissolution of the copolymer according to the invention, and then cooled to ambient temperature with the same stirring. 8.06 g of tebuconazole (solid active agent) are subsequently added, followed by homogenization with a spatula. The mixture is then subjected to 3 minutes in an Ultra Turrax T50 with a dual effect blade of D=45 mm, at 6000 rpm, before being subjected to wet milling in a MiniMotor Mill of the Eiger Torrance LTD brand, with a 50 ml capacity.

38 ml of SEPR ER 120 A, ZrO₂/SiO₂ beads 0.8 to 1.25 mm in diameter are used for the milling, which lasts 10 min at 3300 rpm (graduation 7).

It is verified under an optical microscope that all the particles are less than 10 μm in size.

TABLE 7

| | Content as % by |
|---|---|
| Formulation | weight |
| Tebuconazole (98%) | 6.2 |
| Copolymer of example 1 | 3 |
| Alkamuls VO2003 | 20.00 |
| Rapeseed oil | 70.704 |
| Aerosil 200 | 0.046 |
| Sodium carbonate | 0.09 |

Various “standard” trade tests for agrochemical formulations are carried out on this formulation according to table 8 in order to be sure of the quality and the stability of said formulation.

Particularly of interest is the stability of the dispersion in oil (phase separation: syneresis and/or sedimentation) during aging tests (ambient temperature, 0° C. for 7 days, 54° C. for 15 days) and also its viscosity. Also of interest is the behavior of the dispersion in oil during its switching in water (pH, wet sieve residue, foam and stability of the emulsion generated at various times (0, 0.5 h, 24 h and redispersion at 24 h followed by wait of 0.5 h)).

The results are presented in table 8:

TABLE 8

| | | | Composition of table 7 | | |
|---|---|---|---|---|---|
| | | | Storage | | |
| | | | 20 days at ambien | | 15 days at |
| | | | temperature | 7 days at 0° C. | 54° C. |
| Syneresis | | | traces | 0% | 0% (changing |
| | | | | | color and color |
| | | | | | gradient on |
| | | | | | 5%) |
| Flowability | | | Flowable | Flowable | Flowable |
| Viscosity | Brookfield Viscosity (20 rpm) | | 800(25° C.) | 600(24° C.) | 1300(24° C.) |
| (mPa s⁻¹) | | | | | |
| pH (at 5%) | pH | | 8.4 | 8.4 | 8.4 |
| Wet sieve | Retention on 80 μm | | 0% | 0% | 0% |
| residue | sieve for 5% of | | | | |
| (inspiration | composition diluted in | | | | |
| taken from MT | mains water after | | | | |
| 185) | magnetic stirring | | | | |
| Foam (inspiration | Use of a 100 ml | | 0 immediate | 0 immediate | 0 immediate |
| taken from MT 47.2) | graduated cylinder, | | | | |
| | addition of 1 ml of | | | | |
| | composition to mains | | | | |
| | water, 30 inversions | | | | |
| | and observation of the | | | | |
| | amount of foam | | | | |
| Dispersion stability (inspiration taken from | Waiting time before | | | | |
| MT 180) | characterization | | | | |
| Formulation diluted to | 0 h | Appearance of | White | White emulsion. | Slightly clearer |
| 1%, at 30 +/− 2° C., in CIPAC D water. | | the emulsion | emulsion. | | white emulsion |
| 100 ml graduated cylinder used, inversion | 0.5 h | Cream (mm) | 0 | 0 | 0 |
| 10 times, then obsevation after: | | Oil (mm) | 0 | 0 | 0 |
| | | Sediment (mm) | 0 | 0 | 0 |
| | 24 h | Cream (mm) | traces | traces | 1.5 |
| | | Oil (mm) | 0 | 0 | 0 |
| | | Sediment (mm) | 0.1 | 0.1 | 0 |
| | | Redispersion | OK | OK | OK |
| | 24.5 h | Cream (mm) | 0 | 0 | 0 |
| | | Oil (mm) | 0 | 0 | 0 |
| | | Sediment (mm) | 0 | 0 | 0 |

The results show a formulation that is stable at ambient temperature after 20 days and at 0° C. after 7 days since no phase separation appears (nor syneresis, nor sedimentation). The accelerated aging test (15 days at 54° C.) shows a change in color and in color gradient on the 5% from the top of the sample. The formulation is flowable.

A pH of 8.4 is measured after dilution of 5% by weight of the composition in mains water. The wet sieve residue test is good, since no residue is retained on the sieve. The foam test is excellent, since no residual foam is present after 30 inversions of the graduated cylinder.

The dispersion stability test is good, with a stable emulsion being obtained, said emulsion exhibiting slight creaming after storage at 54° C. and some traces of deposit at 0° C. and at ambient temperature. This cream is redispersible.

These results show that the use of the copolymer according to the invention makes it possible to prepare compositions comprising a dispersion of phytosanitary active agents that are stable over time and at temperature.

Of course, these results could not limit the invention to active agents of this type.

These results demonstrate in fact, more generally, that the copolymers of the invention make it possible to prepare compositions of all types (agrochemical, cosmetic, pharmaceutical, etc.) comprising at least one active agent, which are stable over time and over a wide temperature range.

### EXAMPLE 9

### Evaluation of the Stability of a Complete Formulation Prepared with a Copolymer in Accordance with the Invention

45 g of the copolymer of example 1 are added, with a spatula, to a 1000 ml Pyrex beaker, as are 200 g of emulsifier (polyethylene glycol esters of fatty acids sold by the company Rhodia under the name Alkamuls VO2003) and 710.15 g of rapeseed oil, with a pipette. 0.85 g of Aerosil 200 silica (Evonik) and also 1 g of sodium carbonate are added with a spatula. This mixture is then heated with stirring at 300 rpm until complete dissolution of the copolymer according to the invention, and then cooled to ambient temperature with the same stirring. 1 g Silcolapse RG22 (Rhodia) antifoam is then added with stirring. 43 g of nicosulfuron (solid active agent, purity 98%) are subsequently added, followed by homogenization with a spatula. The mixture is then subjected to 3 minutes in an Ultra Turrax T50 with a dual effect blade of D=45 mm, at 6000 rpm, before being subjected to wet milling in a Vibro Mac LAB 2T, with a 1000 ml capacity.

800 ml of SEPR ER 120 A, ZrO₂/SiO₂ beads 0.8 to 1.25 mm in diameter are used for the milling, which lasts 20 min.

It is verified under an optical microscope that all the particles are less than 10 μm in size.

TABLE 9

| | Content as % by |
|---|---|
| Formulation | weight |
| Nicosulfuron (98%) | 4.3 |
| Copolymer of example 1 | 4.5 |
| Alkamuls VO2003 | 20.00 |
| Rapeseed oil | 71.015 |
| Aerosil 200 | 0.085 |
| Sodium carbonate | 0.1 |
| Silcolapse RG22 | 0.1 |

Various “standard” trade tests for agrochemical formulations are carried out on this formulation according to table 10 in order to be sure of the quality and the stability of said formulation.

Particularly of interest is the stability of the dispersion in oil (phase separation: syneresis and/or sedimentation) during aging tests (ambient temperature, 0° C. for 7 days, 54° C. for 15 days) and also its viscosity. Also of interest is the behavior of the dispersion in oil during its switching in water (pH, residue and stability of the emulsion generated at various times (0, 1 h)).

The results are presented in table 10:

TABLE 10

| | | | Storage | | |
|---|---|---|---|---|---|
| | | | 30 days at | | |
| | | | ambient | 7 days at | 15 days at |
| | | | temperature | 0° C. | 54° C. |
| Syneresis | | | 0% | 0% | 12% |
| Flowability | | | Flowable | Flowable | Flowable |
| Viscosity (mPa s⁻¹) | Brookfield Viscosity | | 960 (20° C.) | 980 (20° C.) | 1140 (20° C.) |
| | (20 rpm) | | | | |
| pH (at 5%) | pH | | 5.2 | 5.2 | 5.1 |
| MT 180) | Waiting time before | | | | |
| Formulation | characterization | | | | |
| diluted to | 0 h | Appearance of | White | White | White |
| 1%, at 30 +/− | | the emulsion | emulsion. | emulsion. | emulsion |
| 2° C., in CIPAC | 1 h | Cream (mm) | 1 | 1 | 2 |
| D water. | | Oil (mm) | 0 | 0 | 0 |
| 100 ml | | Sediment (mm) | 0 | 0 | 0 |
| graduated | | | | | |
| cylinder | | | | | |
| used, | | | | | |

The results show a formulation that is stable at ambient temperature after 30 days and at 0° C. after 7 days since no phase separation appears (nor syneresis, nor sedimentation). The accelerated aging test (15 days at 54° C.) shows a limited phase separation on the 12% from the top of the sample, which can be rehomogenized after mixing. The formulation is flowable. A pH of 5.2 is measured after dilution of 5% by weight of the composition in CIPAC D water. The dispersion stability test is good, with a stable emulsion being obtained, said emulsion exhibiting slight creaming and some traces of deposit. This cream is redispersible.

These results show that the use of the copolymer according to the invention makes it possible to prepare compositions comprising a dispersion of phytosanitary active agents that are stable over time and at temperature.

### EXAMPLE 10

### Evaluation of the Stability of a Complete Formulation Prepared with a Copolymer in Accordance with the Invention

45 g of the copolymer of example 1 are added, with a spatula, to a 1000 ml Pyrex beaker, as are 200 g of emulsifier (polyethylene glycol esters of fatty acids sold by the company Rhodia under the name Alkamuls VO2003) and 699.15 g of rapeseed oil, with a pipette. 0.85 g of Aerosil 200 silica (Evonik) and also 1 g of sodium carbonate are added with a spatula. This mixture is then heated with stirring at 300 rpm until complete dissolution of the copolymer according to the invention, and then cooled to ambient temperature with the same stirring. 1 g Silcolapse RG22 (Rhodia) antifoam is then added with stirring. 53 g of flufenoxuron (solid active agent, purity 98%) are subsequently added, followed by homogenization with a spatula. The mixture is then subjected to 3 minutes in an Ultra Turrax T50 with a dual effect blade of D=45 mm, at 6000 rpm, before being subjected to wet milling in a Vibro Mac LAB 2T, with a 1000 ml capacity.

800 ml of SEPR ER 120 A, ZrO₂/SiO₂ beads 0.8 to 1.25 mm in diameter are used for the milling, which lasts 20 min.

It is verified under an optical microscope that all the particles are less than 10 μm in size.

TABLE 11

| | Content as % by |
|---|---|
| Formulation | weight |
| Flufenoxuron (98%) | 5.3 |
| Copolymer of example 1 | 4.5 |
| Alkamuls VO2003 | 20.00 |
| Rapeseed oil | 69.915 |
| Aerosil 200 | 0.085 |
| Sodium carbonate | 0.1 |
| Silcolapse RG22 | 0.1 |

Various “standard” trade tests for agrochemical formulations are carried out on this formulation according to table 12 in order to be sure of the quality and the stability of said formulation.

Particularly of interest is the stability of the dispersion in oil (phase separation: syneresis and/or sedimentation) during aging tests (ambient temperature, 0° C. for 7 days, 54° C. for 15 days) and also its viscosity. Also of interest is the behavior of the dispersion in oil during its switching in water (pH, and stability of the emulsion generated at various times (0, 1 h)).

The results are presented in table 12:

TABLE 12

| | | | Storage | | |
|---|---|---|---|---|---|
| | | | 30 days at | | |
| | | | ambient | 7 days at | 15 days at |
| | | | temperature | 0° C. | 54° C. |
| Syneresis | | | 0% | 0% | 2% |
| Flowability | | | Flowable | Flowable | Gel |
| Viscosity (mPa s⁻¹) | Brookfield Viscosity | | 860 (20° C.) | 900 (20° C.) | 6000 (20° C.) |
| | (20 rpm) | | | | |
| pH (at 5%) | pH | | 8.1 | 8.3 | 8.6 |
| Dispersion stability | Waiting time before | | | | |
| (inspiration taken from | characterization | | | | |
| MT 180) | 0 h | Appearance of the | White | White | White |
| Formulation diluted to | | emulsion | emulsion. | emulsion. | emulsion |
| 1%, at 30 +/− 2° C., in | 1 h | Cream (mm) | 0 | 0 | 0 |
| CIPAC D water. | | Oil (mm) | 0 | 0 | 0 |
| 100 ml graduate cylinder | | Sediment (mm) | 0 | 0 | 0 |
| used, inversion 10 times, | | | | | |
| then observation after: | | | | | |

The results show a formulation that is stable at ambient temperature after 30 days and at 0° C. after 7 days since no phase separation appears (nor syneresis, nor sedimentation). The accelerated aging test (15 days at 54° C.) shows a virtually zero phase separation (2% on the top of the sample), nevertheless the formulation appears to be gelled. It is necessary to stir it in order for it to become flowable again, and this resulting formulation remains very viscous. A pH of 8.1 is measured after dilution of 5% by weight of the composition in CIPAC D water. The dispersion stability test is good, with a stable emulsion being obtained, said emulsion exhibiting no creaming, nor any traces of deposit.

These results show that the use of the copolymer according to the invention makes it possible to prepare compositions comprising a dispersion of phytosanitary active agents that are stable over time and at temperature.

### EXAMPLE 11

### Preparation of a Concentrated Liquid Formulation that is Easy to Use, Containing a Copolymer According to the Invention and an Emulsifier

This concentrate can in particular be used for preparing a complete formulation, of OD type for example.

215.3 g of the copolymer of example 1 are added, with a spatula, to a 1000 ml Pyrex beaker, as are 781.3 g of emulsifier (polyethylene glycol esters of fatty acids sold by the company Rhodia under the name Alkamuls VO2003) and 3.4 g of Aerosil 200 silica (Evonik), with a pipette. This mixture is then heated with stirring at 300 rpm until complete dissolution of the copolymer according to the invention, and then cooled to ambient temperature with the same stirring.

TABLE 13

| | Content as % by |
|---|---|
| Formulation | weight |
| Copolymer of example 1 | 21.53 |
| Alkamuls VO2003 | 78.13 |
| Aerosil 200 | 0.34 |

A concentrated liquid formulation which is stable and easy to handle is obtained. This liquid concentrate containing the two functionalities (on the one hand, the rheological agent (copolymer according to the invention) and, on the other hand, the emulsifier (Alkamuls VO2003)) can be stabilized if necessary by adding a little silica. The measured viscosity of this mixture is of the order of 2500 cP at 20 rpm and 25° C. and is stable at ambient temperature, 0° C. and 54° C.

An appropriate amount of this concentrate can be used to prepare, in a second step, a complete formulation, for example of OD type, like those described in examples 8 to 10.

1. A copolymer obtained by radical polymerization of:
a fatty substance (A) comprising unsaturations, optionally further comprising a function having a labile proton; and
at least one monomer (B) comprising:
(a) at least one function that can be polymerized by radical polymerization and (b) at least one linear or branched alkyl chain comprising from 16 to 44 carbon atoms.

2. The copolymer according to claim 1, wherein the monomer (B) comprises a linear or branched alkyl chain comprising 22 carbon atoms or 44 carbon atoms.

3. The copolymer according to claim 1, wherein the fatty substance (A) is selected from mono, di or triglycerides of fatty acids, or methyl or ethyl esters thereof; unsaturated hydrocarbons of which the carbon-based chain comprises at least one double or triple bond and/or which are hydroxylated; fatty acids of which the carbon-based chain comprises at least one double or triple bond and/or which are hydroxylated; fatty alcohols; fatty amines; animal oils or oils of animal origin, or derivatives thereof; silicone oils; terpene compounds; synthetic resins carrying a function having a labile proton; or mixtures thereof.

4. The copolymer according to claim 1, wherein the fatty substance (A) is selected from vegetable oils or oils of vegetable origin, the vegetable oils or oils of vegetable origin comprising:
triglycerides of saturated or unsaturated fatty acids comprising at least 12 carbon atoms; or
esters of triglycerides of saturated or unsaturated fatty acids comprising at least 12 carbon atoms;
or animals oils or oils of animal origin;
or mixtures thereof.

5. The copolymer according to claim 4, wherein the fatty substance (A) is selected from rapeseed oil, soybean oil, corn oil, castor oil, groundnut oil, butter oil, cottonseed oil, linseed oil, coconut oil, olive oil, palm oil, grapeseed oil, copra oil, or mixtures thereof.

6. The copolymer according to claim 1, wherein the monomer (B) is selected from:
alkyl acrylates;
alkyl methacrylates;
alkylacrylamides;
alkylmethacrylamides;
alkyl vinyls, in particular alkyl allyls or alkyl vinyl ethers; or
alkylstyrenes;
in which the alkyl is a linear or branched chain.

7. The copolymer according to claim 6, wherein the monomer (B) is selected from an alkyl acrylate in which the alkyl chain comprises 22 carbon atoms or an alkyl acrylate in which the alkyl chain comprises 44 carbon atoms.

8. The copolymer according to claim 1, wherein the fatty substance (A) is grafted and the degree of grafting of the fatty substance (A) is between 5% and 80%, according to one of the following formulae (eq1), (eq2) or (eq3) depending on whether the fatty substance (A) comprises, respectively, only unsaturations (eq1), only hydroxyl functions (eq2) or both unsaturations and hydroxyl functions (eq3):
$$\begin{matrix}degree of grafting = ( \frac{A 2}{A 1} ) & ( eq 1 ) \\ degree of grafting = ( \frac{B 2}{B 1} ) & ( eq 2 ) \\ degree of grafting = ( \frac{A 2}{A 1} ) + ( \frac{B 2}{B 1} ) & ( eq 3 )\end{matrix}$$
wherein
$$( \frac{A 2}{A 1} ) = ( \frac{number of unsaturations polymerized}{number of initial unsaturations} )$$
$$( \frac{B 2}{B 1} ) = ( \frac{number of hydroxyl functions substituted}{number of initial hydroxyl functions} )$$

9. The copolymer according to claim 1, wherein the molecular weight of the copolymer is between 10 000 and 1×10⁶ g/mol.

10. A process for preparing a copolymer, the process comprising reacting a fatty substance (A) comprising unsaturations, optionally further comprising a function having a labile proton, with at least one monomer (B) comprising at least one function that can be polymerized by radical polymerization and at least one linear or branched alkyl chain comprising from 16 to 44 carbon atoms in the presence of a radical initiator.

11. A process for modifying the rheological properties of a nonaqueous medium, the process comprising adding to a nonaqueous medium a copolymer according to claim 1, thereby modifying the rheological properties of the nonaqueous medium.

12. The process according to claim 11, wherein the rheological properties modified are the presence of a rheological threshold and/or the viscosity and/or the gelling properties.

13. (canceled)

14. (canceled)

15. A composition that is emulsifiable by mixing with water, comprising:
a nonpolar medium;
a compound dispersed within said nonpolar medium;
a copolymer according to claim 1; and
an emulsifier.

16. (canceled)

17. The composition according to claim 15, wherein the nonpolar medium comprises a mixture of triglycerides.

18. (canceled)

19. The composition according to claim 15, wherein the nonpolar medium is a vegetable oil or a mixture of vegetable oils and the emulsifier is a polyethylene glycol ester of fatty acids.

20. (canceled)

21. (canceled)

22. A process for forming an emulsion of oil-in-water type comprising mixing the composition according to claim 15 with an aqueous phase.

23. A concentrated composition comprising a copolymer according to claim 1 and an emulsifier.

24. The copolymer according to claim 1, wherein fatty substance (A) is castor oil, rapeseed oil, or oleic acid; and the at least one monomer (B) is behenyl acrylate.

25. The composition according to claim 15, wherein the nonpolar medium comprises natural triglycerides selected from vegetable oils or oils of vegetable origin, or animal oils or oils of animal origin.

26. The composition according to claim 15, wherein the nonpolar medium comprises vegetable oils or oils of vegetable origin selected from rapeseed oil, soybean oil, corn oil, castor oil, groundnut oil, butter oil, cottonseed oil, linseed oil, coconut oil, olive oil, palm oil, grapeseed oil, copra oil, or mixtures thereof.
